SEQUENCE LISTING (8 sequences)

SEQ ID NO: 1 - DNA, 3176 bp, C. albicans
  CDS (354)...(1914)
cttgcatgcaatgaaaatagtttatcttgaatctctgatttgaatgtagtaaaagtctattgtaaactaaaacttgctccctaaaagctaatcatattcaagccgagacctacaaatacaacttttgaacttgtcacaatcatcgcattctttccaatgtctgtaacgtgtgatgagtcttctaattcatagatctcaaggtcatgtaatcaaaccaaagccatgatatattttcctgcatataaaaaggttggatggtgagaagaagaacaagaagaaaaaaaaaagtctccaaaaatgtcagaactagcttgttgatgggcaaccgttgacttgtttatggccatactgcaatgaatgttggatctattttaaatgacgacccaccatcaagtgggaatgcgaatgggaatgatgataataccaagattattaaatcccctactgcataccataaaccttctgttcatgaacgtcattcaataacgagcatgttgaatgacactccgtcagattcaactccaactaaaaaaccagaaccgactataagtccagagtttagaaaacccagcataagtctgttaacttctccaagtgttgcacataaacctccgccactaccaccgtcactgagtctggttggaagtagtgagcattcgagtgcaagatcgtccccggctatcacgaagagaaactcgattgcaaacattatcgatgcttatgaagaaccagctactaaaactgaaaaaaaggctgagctaaactcaccaaagataaaccaactgacaccggtgccaaagcttgaggaacacgagaatgatacaaacaaagtagaaaaggttgtggatagtgcacctgaaccaaaaccaaaaaaggagcctcaaccagtttttgacgaccaagacgatgacttgacaaaaatcaaaaagctcaagcaatctaagaaaccacgtcggtatgaaacacctccaatttgggcccagaggtgggttcccccaaatagacagaaggaggaaactaatgttgatgacgggaatgaagccataactagactttctgaaaaaccggtatttgattataccactaccagaagtgttgatttggagtgtagtattactggtatgatacccccaagttcaatcacgagaaaaatagctgaatgggtgtatgccaatttttccaatgttgaagaaaaaagtaaaaggaatgttgaattggagttgaaatttgggaaaattattgacaaaagaagtggtaatagaattgacttgaatgtggtgacagaatgtattttcactgatcattctagtgtgttttttgacatgcaagtggaagaggtggcctggaaagaaataacaaaattcttggatgaattggaaaaaagtttccaagaagggaaaaagggaagaaaatttaaaactcttgaatctgataatactgacagtttctatcaattggggagaaaaggtgagcaccctaagcggattcgtgtaaccaaagacaacttactatcgccaccgagattggttgccatacagaaggaacgtgtggcagatttatatattcacaatccgggctccttatttgatttgaggttatctatgtcattggaaataccagtgccacaggggaacattgagtcgattattaccaagaataagccagagatggtcagggagaagaagagaatttcttatacacatccacctaccattaccaaatttgacttgactagggtcattggtaataaaacagaagataaatatgaggtagagttggaggcgggtgttatggaaatatttgctgctattgataaaatccagaaaggggtagataatcttagattggaggaattaattgaagtttttttgaacaatgcaagaactctcaataatagattgaacaagatttgctagcaggacttcctgacctgttatgatgtaatgtagtgtatcatttgtaatcctagaatcagtctttaataataaaaaataatatcaggggtgttctttgaattgataaagttatttacaggcataaataggccaatatttgcaaagtctgtattcgattaagcaaaaataaatagctgtataatgttgaaaaaaaaaaatattgactgaaacgagattcgaactcgcgcccactttcgtagaccaggattgttgtggtaaacgttacgcgataagatttaccgccgccttaccctggcgccttgaccgctcggccatccagtcacgtgagaagagctaactgtggtaagctttttacaactcgaccaaacaactcttttcatgttgaaaccataaatccacttatttatatagggcttataattcaaaacccacactatcatttacgtcttgttttgctgatttatatataattataggttggataagtatcaaatatgagcctacttggacgtagagtttttcatttaatccaattgaagagataatagagaggacattctaagatcagttttttgcatcaacattatctccaagttttcaaggaaataagttttaacaaagagttgacaggaatttgctcgggaaaagaggtaacatccaatcatattacttaagcggcacataaaaacagaccacctctaaaacaaagagttagtatttggttttccaagtactaatacactttccacctcttgttaacaaaatattataattcagttgcgactttacaaacagcatgatattatatctttcctcttaatctgttctctcctatttcaataacaattgagcgttagacatgaccagagaataattcacaaattcaagacgacaaagaagtgtagattattcagaacatggtaagtgtttcctcaaaacttacccgtttaacggaacacactcattaacataagccttggcgtcagtttttaaaattccacatctgccaattctctcgtaccacagtgtttgatattttacatccatccttcgtcgtttaatgtgagcttttaaaagtttgagattcttgtgctgacactaactttgccgcaatcaagtgatttctatttttggttttcttggttgagcacctcgcacattattttttggttttcttgtcaattcgtgataaaagacttgccatatgtctatttccacttctacgtacaacaattcaattccaaattatgtggttctaga

SEQ ID NO: 2 - protein, 520 aa, C. albicans
MNVGSILNDDPPSSGNANGNDDNTKIIKSPTAYHKPSVHERHSITSMLNDTPSDSTPTKKPEPTISPEFRKPSISLLTSPSVAHKPPPLPPSLSLVGSSEHSSARSSPAITKRNSIANIIDAYEEPATKTEKKAELNSPKINQLTPVPKLEEHENDTNKVEKVVDSAPEPKPKKEPQPVFDDQDDDLTKIKKLKQSKKPRRYETPPIWAQRWVPPNRQKEETNVDDGNEAITRLSEKPVFDYTTTRSVDLECSITGMIPPSSITRKIAEWVYANFSNVEEKSKRNVELELKFGKIIDKRSGNRIDLNVVTECIFTDHSSVFFDMQVEEVAWKEITKFLDELEKSFQEGKKGRKFKTLESDNTDSFYQLGRKGEHPKRIRVTKDNLLSPPRLVAIQKERVADLYIHNPGSLFDLRLSMSLEIPVPQGNIESIITKNKPEMVREKKRISYTHPPTITKFDLTRVIGNKTEDKYEVELEAGVMEIFAAIDKIQKGVDNLRLEELIEVFLNNARTLNNRLNKIC

SEQ ID NO: 3 - DNA, 2428 bp, C. albicans
  CDS (201)...(1625)
gagctcattatgttcctaacaaaaaagactttcttgaaccaattgtaaagttatttgacaaaacgttgaatgaaaagccaatgttatagatgattttatagagtaaaatagataaattgttatgttgtaaggaaaaaaaaaaagcgagcaaaaaaaaaaattgagttgaaacatgaatcattagtttctacaccactagcatgtctaccgattcgtacactccctcacaagagcctggttcaaagcgtttaaagacgggcgaatctgtatttgcaagaagaggagtatcgccaagtactggtggagtggcatcagcctacggtaatgagagtgagaaaaagccatcgtggttacaaaccaacaaaagtgatattgatgggaagtacgataaatatggagagagaagaaatgcccatactacaacaagagactcaagacttgataggttaaagcgagttcgtcaaaagctggctgagcgggaagatgttggtcatgaaggagacgaaggagacgaggatgagggtatattaccttatattcatttacaagcggccaaccctgccatcattcacaacgagaaacaggaaaactatcgtacgtttcagagtaggatatcgaatagagagaatagagacatcaatagtattgtgagggcacactataatcagcgaacacaacaagcaaaacagcaaggatcccgagtcaattcgccaatttacaaaatgaggaatttcaacaatgccattaaatacatattgttgggtaattgggccaaacataatccagaggaattggatttgttttcttttttggatttatgttgtggcaaaggtggggatttgaacaaatgccaatttattggcattgatcaatatattggcattgacattgctgatttatcggtcaaagaagcatttgaacggtacacaaaacaaaaggcgaggttcagacactctaatcagaattctaatcggtatacttttgaggcgtgttttgccacaggggattgtttcacccaatttgtgcctgatatcctagagccaaatttccctggaattatagaacgtgcatttcccgtggatattgtttccgcccagttttcgttgcattattcttttgaaagtgaagaaaaggtacgtacattgttgaccaacgtcacaaggtcgttgcgttcaggaggcacttttattggcacaattccttcctctgatttcataaaggcaaaaatagttgacaaacatttgcaacgagatgaaaaggggaaagcgaagtttggtaatagtttgtattcggtgacgtttgaaaaagatcctccagaagatggcgtattccgtcctgcgtttgggaacaagtacaattattggttgaaagatgccgttgacaatgttcctgagtatgtggttccgtttgaaacattgagatcattgtgtgaagagtacgatttggttttgaagtataaaaagagttttacagatatattcaaccaggagattccaaagtattttagtaaattgaataaaaatctaattgatggaatgaaacgaagtgatggcaagtacggtgctgaaggtgacgaaaaggaagcagtggcattttacataggatttgtatttgagaaggtataggatatgtctgggtagtgtgtagcattttttgggtcaaggatctttacgaaaaaattaagaaaagtaaacaacagaggactttttccgatccgttttagtcaatgactcgctgtacataaaaccaatctaaatggttaagtaccactaaaaaaaaaaaaaaaactccatctatatttattttcagaacaattacttataactaaaagtaatcggactatataagtcgaggcaacacaaagtttaggcgtgatagtagttcgactagatactctattccttttttttcccaccttgcagtcaaatagacgaaaaatttgatagcgattatatttgctagtcgtatattgaagctccatattagatactatggcagaccaaagcaccttaacctttatagtgtctccagtttaagccctggtacaagcgagataataaaaagttagtcgatggcagatctgaatttagaagagccaaattgcatatgtacaataaatattatataatgtgaggtgacgggcaaaaataaaattatcttttcgtaatgtcacgtctgtctaggtaacattcttcctgcacttcacagccttatgcaatacaatcatcaaattatacattgtcgaaccaaaatgatgacaatgtacctaggtacacaattttcttgttttttttttctattttcgtcgtgtgttcatgaacaatcagaaccccattgtcaaatagatataactcgtgagaagaaataattcttgagttagtaatggatttgtgcaatagttccccctttgcaaaataggtttactagt

SEQ ID NO: 4 - protein, 474 aa, C. albicans
MSTDSYTPSQEPGSKRLKTGESVFARRGVSPSTGGVASAYGNESEKKPSWLQTNKSDIDGKYDKYGERRNAHTTTRDSRLDRLKRVRQKLAEREDVGHEGDEGDEDEGILPYIHLQAANPAIIHNEKQENYRTFQSRISNRENRDINSIVRAHYNQRTQQAKQQGSRVNSPIYKMRNFNNAIKYILLGNWAKHNPEELDLFSFLDLCCGKGGDLNKCQFIGIDQYIGIDIADLSVKEAFERYTKQKARFRHSNQNSNRYTFEACFATGDCFTQFVPDILEPNFPGIIERAFPVDIVSAQFSLHYSFESEEKVRTLLTNVTRSLRSGGTFIGTIPSSDFIKAKIVDKHLQRDEKGKAKFGNSLYSVTFEKDPPEDGVFRPAFGNKYNYWLKDAVDNVPEYVVPFETLRSLCEEYDLVLKYKKSFTDIFNQEIPKYFSKLNKNLIDGMKRSDGKYGAEGDEKEAVAFYIGFVFEKV

SEQ ID NO: 5 - DNA, 17 bp, Artificial Sequence
  Primer
gggcatgcaagtggaag

SEQ ID NO: 6 - DNA, 19 bp, Artificial Sequence
  Primer
gggtacccaatgaccctag

SEQ ID NO: 7 - DNA, 23 bp, Artificial Sequence
  Primer
gggcatgcaatgttcctgagtat

SEQ ID NO: 8 - DNA, 21 bp, Artificial Sequence
  Primer
gggtaccaatgcnacngcttc

(19) United States
(12) Patent Application Publication
(10) Pub. No.: US 2002/0018774 A1
(43) Pub. Date: Feb. 14, 2002

(54) CANDIDA ALBICANS mRNA 5'5-TRIPHOSPHATASE (CET-1) POLYNUCLEOTIDES

(76) Inventors: SIMON GREEN, PLEASANTON, CA (US); GARY DALLMANN, MENLO PARK, CA (US); MAGDELEINE HUNG, HAYWARD, CA (US); JULIE LANE, OAKLAND, CA (US); CHARLES M. MOEHLE, CASTRO VALLEY, CA (US)

Correspondence Address: HELLER EHRMAN WHITE & MCAULIFFE LLP, 1666 K STREET,NW, SUITE 300, WASHINGTON, DC, 20006, US

(21) Appl. No.: 09/213,678

(22) Filed: Dec. 17, 1998

(51) Int. Cl. G01N 33/53; C12N 5/00; C12N 15/70; C12P 21/06; A61K 39/395; C12N 15/74; C12N 15/63; C12N 15/09; G01N 33/569; C07H 21/04; C12P 21/04; C12N 15/00; C12N 5/02

(52) U.S. Cl. 424/130.1; 536/23.5; 435/320.1; 435/325; 435/71.2; 435/69.1; 435/7.1; 435/975

(57) ABSTRACT

The present invention relates to enzymes involved in capping of fungal mRNAs, and molecules that inhibit such enzymes. In particular, the invention relates to the novel C. albicans capping enzyme genes ABD1 and CET1 and their encoded protein products, as well as derivatives and analogs thereof. The invention also relates to methods of using of fungal capping enzymes to screen for fungal inhibitors.

Work described herein was supported in part by a SBIR grant from the National Institutes of Allergies and Infectious Diseases. The United States government may have rights to certain aspects of the invention described herein.

### 1. FIELD OF THE INVENTION

The present invention relates to enzymes involved in capping of fungal mRNAs, and molecules that inhibit such enzymes. In particular, the invention relates to the novel C. albicans capping enzyme genes ABD1 and CET1 and their encoded protein products, as well as derivatives and analogs thereof. The invention also relates to methods of using of these enzymes to screen for fungal inhibitors.

### 2. BACKGROUND OF THE INVENTION

2.1 Significance of Fungi as Pathogenic Organisms

Fungal pathogens are responsible for a variety of diseases in humans and animals ranging from mycoses involving skin, hair, or mucous membranes to severe systemic infections, many of which are fatal. In recent years there has been a marked increase in the number of serious fungal infections as a result of the growing number of immunosuppressed and immunocompromised individuals.

For example, fungal infections represent a major problem in patients with AIDS. Indeed, the appearance in the early 1980's of rare opportunistic fungal infections and malignancies was a harbinger of the AIDS pandemic. Many of the infections seen in AIDS patients are also observed in other patients who are immunocompromised, including transplant patients on immunosuppressive drugs and cancer patients (Rosenberg and Brown, 1993, Disease-a-month 39, 507-569).

In cancer patients, neutropenia, T-cell defects, B-cell defects, and spleenectomy can all increase susceptibility to opportunistic infection. Defects in the skin or mucous membranes accompanying treatment and the use of catheters in patient care are additional contributing factors. An increased susceptibility to fungal infections also arises from treatment with broad-spectrum antibiotics, severe diabetes, invasive procedures such as intravascular catheterization, administration of parenteral nutrition, addiction to intravenous drugs and prosthetic implants. The most common fungi associated with opportunistic infections are, Candida spp., Aspergillus spp., Cryptococcus neoformans and Pneumocystis carinii. Candida albicans is by far the major opportunistic pathogen; however the frequency of non-albicans Candida spp. is increasing. Many Candida spp. can cause oral thrush, esophagitis, urinary tract infections, cutaneous or ocular lesions, meningitis, or endocarditis. Additionally, Candida spp. are now the fourth most common cause of nosocomial infections, accounting for 8-15% of all hospital-acquired bloodstream infections.

Less common organisms associated with opportunistic infection include species of Pneumocystis, Histoplasma, Coccidioides, Mucor, Rhizopus, Trichosporon, Fusarium, Geotrichium, Pseudallescheria, Penicillium, Curvularia and Cunninghamella. As a final example of the vulnerability of immunocompromised individuals to fungal infections, even Saccharomyces (bakers' yeast) has been implicated as an opportunistic human pathogen.

2.2 Current Efforts in Anti-Fungal Drug Discovery

The development of antifungal drug therapies has not evolved as rapidly as the development of antibacterial drug therapies in large part because the human or animal host and the fungal pathogen are both eukaryotes and have many molecular targets in common (recently reviewed in Georgopapadakou, N. H. and Walsh, T. J. (1996) Antimicrob. Agents Chemother. 40, 279-291). To date, most antifungal drugs and lead compounds have been active against components of the fungal cell surface or membrane, and the preponderance of these are active against ergosterol, a fungal-specific sterol, or ergosterol biosynthesis.

For example, polyene macrolides bind to ergosterol. The current “gold standard” of the antifungal polyene macrolides is Amphotericin B. However, it has both short-term and long-term adverse effects, ranging from nausea and vomiting to kidney damage.

Azole-containing and allylamine drugs inhibit lanosterol C14-demethylase and squalene epoxidase, respectively, which are two ergosterol biosynthetic enzymes. Azole drugs such as clotrimazole and miconazole have such adverse side effects that their use is generally limited to the treatment of topical or superficial infections. Imidazole drugs, such as ketoconazole, lack sufficient specificity for the fungal target (cytochrome P-450) and therefore have adverse effects on the human host (e.g., adverse reactions with other drugs and altered steroid metabolism). Fluconazole one of the more recently developed triazole drugs has the advantages of being orally active and causing fewer side effects. However, fluconazole is only fungistatic and not very effective at curing some infections, especially cryptococcal meningitis and aspergillosis.

While there has been some effort directed at intracellular targets, such as folate and nucleotide metabolism, these compounds, e.g., trimethoprim/sulfamethoxazole and fluorocytosine, have problems with toxicity and the occurrence of resistant strains.

Given the limited number of molecular targets currently exploited, it is reasonable to predict the emergence of pathogens that are virtually invincible against the present battery of antifungal treatments. Evidence exists already for the emergence of drug-resistant pathogens showing some cross-resistance to other drugs targeted against ergosterol synthesis (He et al., 1994, Antimicrob. Agents Chemother. 38:2495-2497). Not surprisingly, reports of infections by resistant fungi are on the rise (Id.). It is possible to combat resistance through alternation of antifungal treatments or the use of mixtures of antifungal agents. Needless to say, in order to prevent or delay the buildup of a resistant pathogen population, different therapeutics that are effective against a particular disease must be available.

The diversity of new antifungal therapeutics currently in the development pipeline is extremely limited. The two main areas of research have focused on modifications of the currently available drugs. New formulations of amphotericin B have recently become available in which it is complexed with a heterologous mixture of lipid molecules. As a result, these lipid complexes initially bypass the kidney where amphotericin's toxicity is most evident. These new formulations do have improved toxicity profiles, however, the activity against fungi has not been enhanced so no dramatic improvement in efficacy has been demonstrated in the clinic. Correspondingly, a range of new azole derivatives are being pursued, although some appear to have improved antifungal activity there is the question of cross resistance as a result of prior treatment with fluconazole.

Surprisingly little progress has been made towards the development of antifungal agents against novel therapeutic targets. The most advanced programs are based on echinocandins and nikkomycins which also target components of the cell wall, β-(1-3)-glucan synthase and chitin synthase respectively. These two classes of novel antifungal therapeutics are only now entering the first stages of clinical trials (Georgopapadakou and Walsh, supra). Taking into account these limited recent advances there is still an ongoing need for novel antifungal drugs that are targeted against a wider range of molecular targets, have few side effects, and are effective against pathogens for which current drugs are inadequate.

2.3 Messenger RNA Capping as a Target for Drug Discovery

In all eukaryotic organisms, including humans and fungi, the nuclear DNA encodes genetic information that directs the production of proteins. DNA is used as a template by RNA polymerase II (RNA pol II) to produce messenger RNA (mRNA) transcripts. These mRNA transcripts are then used, in turn, as a template for translation and synthesis of proteins.

Eukaryotic cellular RNA transcripts are modified by the co-transcriptional addition of a cap structure at the 5′ end of the mRNA (FIG. 1). The cap structure is composed of a N-7-methyl-guanylate residue linked to the 5′ methylene group of the ribose of the first nucleotide of an RNA molecule via an unusual 5′-5′triphosphoanyhydride linkage (see FIG. 1). Synthesis of the cap is ubiquitous among eukaryotes and many eukaryotic viruses (Shuman, 1995, Prog. Nucleic Acid Res. Mol. Biol. 50, 101-129). The cap structure is critical for the production of a legitimate RNA template that can be used to direct the synthesis of a functional protein, and is involved in mRNA stability. Other essential processes that require a properly capped RNA are 3′ end processing and poly adenylation, pre-mRNA splicing and mRNA transport from the nucleus to the cytoplasm. Indeed, a knockout of any one of the genes necessary for RNA capping is lethal in S. cerevisiae. Hence, RNA capping is an essential cellular process.

Distinct evolutionary differences exist between the capping enzyme systems of viruses, fungi and metazoans. Three enzymatic activities are required for cap synthesis: a triphosphatase (TP'ase); a guanylyltransferase (GT'ase); and a methyltransferase (MT'ase). In vaccinia virus all three of these activities are contained in a single 95 kDa protein. In fungi these three activities are on separate protein subunits. Capping systems of metazoans contain two protein subunits: a separate MT'ase protein; and a protein containing both the GT'ase and TP'ase activities (FIG. 2).

The following eukaryotic capping enzymes have been cloned and published:

S. cerevisiae guanylyltransferase (CEG1)

Shibagaki et al. (1992) J. Biol. Chem. 267:9521-9528;

S. cerevisiae methyltransferase (ABD1)

Mao et al. (1995) Mol Cell Biol 15:4167-4174;

S. cerevisiae triphosphatase (CET1)

Tsukamoto et al. (1997) Biochem. Biophys. Res. Commun. 239:116-122;

C.albicans guanylyltransferase (CGT1)

Yamada-Okabe et al. (1996) Microbiology 142:2515-2523;

Human capping enzyme (HCE) and mouse capping enzyme

Yue et al. (1997) Proc. Natl. Acad. Sci. USA 94:12898-12903;

Human methyltransferase Ishikawa et al. (1997)

Unpublished but submitted to Genbank (#AB007858);

C. elegans capping enzyme

Takagi et al. (1997) Cell 89:867-873;

C. elegans MT'ase

Wang and Shuman (1997) J. Biol. Chem. 272:14683-14689.

Citation of references hereinabove shall not be construed as an admission that such references are prior art to the present invention.

The deduced protein sequences of the S. cerevisiae GT'ase (CEG1) and the C. albicans GT'ase (CGT1) both encode 52 kD proteins that exhibit 40% identity, 75% homology (42). Hereinafter, the fungal GT'ase gene will be referred to as CGT1. Protein sequence comparison of the fungal GT'ases with the known viral GT'ases revealed a sequence conservation that was limited to 6 relatively short segments (approximately 13 residues each) (Shuman et al., 1994, Proc. Natl. Acad. Sci. USA 91:12046-12050; Fresco et al., 1994, Proc. Natl. Acad. Sci. USA 91:6624-6628). One highly conserved region (-KXDG-) likely contains the active site lysine involved in the formation of the covalent enzyme-GMP intermediate (Hakansson et al. (1997) Cell 89:545-553). These six conserved segments all participated in the GTP binding site of the enzyme. Protein sequences outside of these conserved active site regions were very divergent between fungi and virus.

The CET1 gene coding for the S. cerevisiae TP'ase protein encodes a predicted 62 kDa protein runs aberrantly on SDS-polyacrylamide gels at about 80 kDa, and displays 5′ RNA triphosphatase activity when overexpressed and purified from E. coli. (Tsukamoto et al., supra).

Metazoan capping enzymes contain both TP'ase and GT'ase activities on the same purified protein. The C. elegans gene encodes a 61 kDa protein; the human and mouse genes both encode 68 kDa proteins (Yue et al., supra). The C-termini of these metazoan capping proteins (past residue 200) are homologous to the fungal and viral GT'ases around the active site lysine noted above. The N-terminal 200 residues of the C. elegans, mouse and human capping enzymes all show significant homology to protein tyrosine phosphatases (Fauman and Saper (1996) TIBS 21:413-417) and, when expressed and purified from E. coli, exhibit specific 5′ RNA triphosphatase activity. Additionally, the metazoan TP'ase domains all contain an essential cysteine residue in the active site common to tyrosine phosphatases.

However, the ˜20 kDa metazoan TP'ase domain encoded by about the first 200 residues of metazoan capping enzymes is considerably smaller than the TP'ase subunit in the purified S. cerevisiae capping holoenzyme. Significantly, the S. cerevisiae TP'ase shows no sequence homology to the C. elegans, human or mouse TP'ase domains or to any tyrosine phosphatase (Yue et al., supra).

The S. cerevisiae MT'ase gene (ABD1) has also been cloned and encodes a 50 kDa protein (Mao et al., supra). The gene encoding a metazoan cap MT'ase from C. elegans has been published (Wang and Shuman, supra). This gene encodes a 46 kDa protein with 30% sequence identity and 56% homology to the S. cerevisiae MT'ase.

Additionally, biochemical differences in the mechanisms employed by capping enzymes exist. The fact that the C. elegans, human and mouse TP'ase domain share significant homology with protein tyrosine phosphatases has clarified previous biochemical observations. In assays of the purified rat liver and brine shrimp capping enzyme preparations it was observed that TP'ase activity was optimal in the absence of divalent cations; the presence of divalent cations was inhibitory (Yagi et al. (1984) J. Biol. Chem. 259:4695-4698). A hallmark of protein tyrosine phosphatases is a lack of dependence on divalent cations. In contrast, the TP'ase activity of the purified S. cerevisiae capping holoenzyme required the presence of divalent cations in the assay buffer (Itoh et al., 1984, J. Biol. Chem. 259:13930-13936).

Protein tyrosine phosphatases contain a conserved —CX₅R— active site motif and hydrolyze phosphates via an enzyme-Pi covalent phosphocysteine intermediate. This conserved motif is also present in the TP'ase domains of the C. elegans, human and mouse enzymes, implying a similar mechanism (Yue et al., supra). The S. cerevisiae TP'ase does not contain this motif and likely uses a different mechanism for phosphate hydrolysis.

This structural and biochemical diversity, especially between the fungal and metazoan TP'ase and GT'ase (i.e. subunit vs. single protein, and differences in reaction mechanism and divalent cation requirements) makes the process of RNA capping an attractive antifungal target.

### 3. SUMMARY OF THE INVENTION

The present invention relates to novel fungal capping enzymes TP'ase and MT'ase. Accordingly, the invention provides nucleotide sequences of C. albicans capping enzyme genes CET1 (the TP'ase encoding gene) and ABD1 (the MT'ase encoding gene), and amino acid sequences of their encoded proteins, as well as derivatives (e.g., fragments) and analogs thereof. Nucleic acids hybridizable to or complementary to the foregoing nucleotide sequences are also provided, as are expression vectors containing such polynucleotides, genetically-engineered host cells containing such polynucleotides, CET1 and ABD1 polypeptides, CET1 and ABD1 fusion proteins, therapeutic compositions, CET1 and ABD1 domain mutants, and antibodies specific for CET1 or ABD1. Additionally, a wide variety of uses are encompassed by the invention, including but not limited to, methods of screening for fungal inhibitors using such CET1 and/or ABD1 polypeptides, including but not limited to any combination of CET1, ABD1 and CGT1.

The invention is based, in part, on Applicants' discovery of the C. albicans capping enzyme genes CET1 and ABD1. The CET1 gene encodes a protein with TP'ase activity that is essential for fungal capping of mRNAs. This novel TP'ase protein is 27% identical at the amino acid level to the S. cerevisiae TP'ase capping enzyme. However, neither of these fungal TP'ases show any homology to known metazoan capping enzymes. The C. albicans ABD1 gene encodes the fungal capping enzyme MT'ase. The activities of both of these genes are required for fungal viability.

### 4. BRIEF DESCRIPTION OF THE FIGURES

FIG. 1 illustrates the chemical structure of an mRNA cap.

FIG. 2 is a schematic outline of the mRNA capping reactions.

FIG. 3 shows a diagram of a method using scintillation proximity technology to assay the efficiency of in vitro capping reactions.

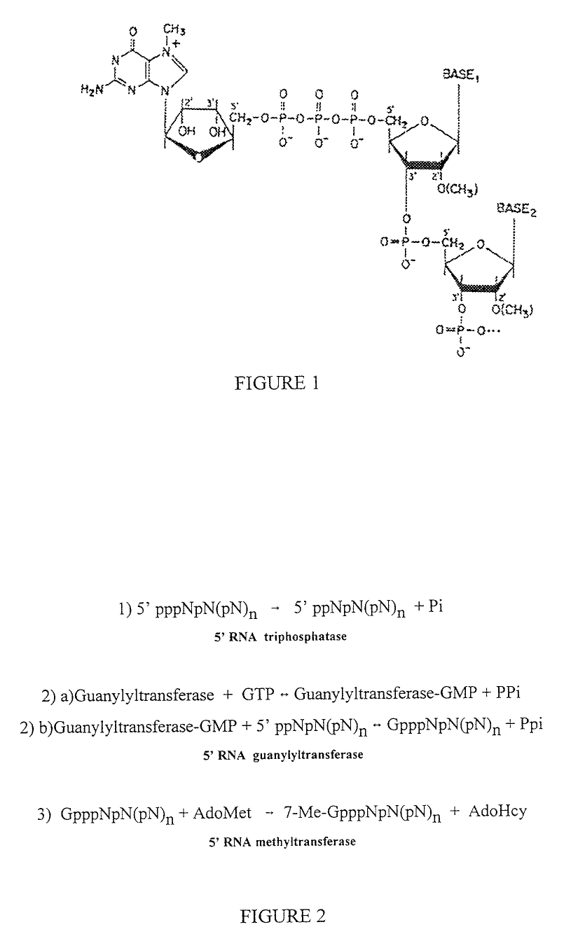

### 5. DETAILED DESCRIPTION OF THE INVENTION

The present invention generally relates to fungal mRNA capping enzymes, the genes encoding them, and methods of using such fungal capping enzymes for both commercial uses and, more particularly, drug discovery. mRNA capping reactions are, for the purposes of the present invention, any of the three reactions illustrated in FIG. 2.

For clarity of discussion, the invention is described below by way of example for the C. albicans CET1 and ADD1 genes and their encoded products. However, the findings disclosed herein can be analogously applied to other homologous members of the C. albicans CET1 and ABD1 family in C. albicans and other fungal species. Thus, the invention encompasses methods of identifying homologous genes in other fungal species. Methods of production of the CET1 and ABD1 proteins, homologs, derivatives and analogs, e.g., by recombinant means, are also provided.

Antibodies to CET1 and/or ABD1, and antibody derivatives and analogs, are additionally provided.

Yet another aspect of the invention provides methods of screening for agents that affect (either increase or decrease) fungal capping and/or fungal translation. In a specific embodiment, these methods make use of the CET1 and/or ABD1 gene products. The invention also relates to a method of identifying genes whose products interact with CET1 and/or ABD1.

5.1 Novel Fungal Capping Enzymes

5.1.1 CET1

Provided herein is the complete C. albicans mRNA triphosphatase gene CET1 (SEQ ID NO:1) and deduced amino acid sequence (SEQ ID NO:2).

By analogy with deletion studies of the S. cerevisiae CET1 protein (see Tsukamoto et al., 1997, Biochem. Biophys. Res. Comm. 239:116-122), the enzymatic activity and protein interaction domain of C. albicans CET1 protein resides in the carboxy-terminal portion of the protein from about amino acid residue 173 to 520. In particular, the sequence PIWAQXWXP from amino acid residues 206 to 214 of SEQ ID NO:2 can define a GT'ase interaction domain of the CET1 protein. Additionally, three triphosphatase motifs occur from amino acids 283-297, 438-451, and 464-476 of SEQ ID NO:2; each of these domains is likely involved in the catalytic site of this enzyme.

For purposes of the invention, functional activities of the CET1 polypeptides include but are not limited to polynucleotide 5′-triphosphatase activity, ability to interact with [or compete for interaction with] CGT1 protein and/or RNA templates, ability to stimulate CGT1 protein activity, antigenicity [ability to immunospecifically bind (or compete with CET1 for binding) to an anti-CET1 antibody], immunogenicity (ability to generate antibody that binds to CETi), and ability to complement a CET1 knockout.

5.1.2 ABD1

The C. albicans mRNA methyltransferase gene ABD1 and deduced amino acid sequence are provided herein, for the first time, in SEQ ID NO:3 and SEQ ID NO:4, respectively. The isolated ABD1 gene sequence (SEQ ID NO:3) encodes a deduced translation product of 474 amino acids (SEQ ID NO:4). By alignment with the genes encoding methyltransferases from S. cerevisiae and Homo sapien (see Wang et al., 1997, J. Biol. Chem. 272:14683-14689 and Pillutla et al., 1998, J. Biol. Chem. 273:21443-21446), the core domain of ABD1 required for enzymatic activity of the C. albicans protein resides in amino acid residues 158 to 474 of SEQ ID NO:4. However, the portion of C. albicans ABD1 needed for fungal cell viability resides in amino acid residues 138-474 of SEQ ID NO:4. Accordingly, the region of amino acids 138 to 158 encompasses a domain involved in interacting with other cellular components (e.g. triphosphatase and/or guanylyltransferase and/or RNA polymerase II). Additionally, amino acid residues 203 to 217 contain a motif involved in binding the AdoMet substrate (see FIG. 2).

For purposes of the invention, functional activities of the ABD1 polypeptides include but are not limited to methyltransferase activity (i.e., addition of a methyl group to a terminal guanine on an RNA template), ability to interact with [or compete for interaction with] RNA templates, guanylyltransferase and/or triphosphatase, antigenicity [ability to immunospecifically bind (or compete with ABD1 for binding) to an anti-ABD1 antibody], immunogenicity (ability to generate antibody that binds to ABD1), stimulation of CET1 and/or CGT1 activity, and complementation of an ABD1 knockout.

5.1.3 Isolation of the CET1 and ABD1 Coding Sequences

The present invention relates to nucleotide sequences of fungal capping enzymes CET1 (the TP'ase) and ABD1 (the MT'ase), and amino acid sequences of their encoded proteins. Also included within the scope of the invention are fragments and other derivatives, and analogs, of the CET1 and ABD1 proteins, and the nucleic acids encoding such fragments or derivatives. The ABD1 and CET1 genes and proteins of the invention include C. albicans CET1 and ABD1 and highly related genes (homologs) in C. albicans and other fungal species. By highly related gene (homolog) of the C. albicans CET1 is meant homologs encoding proteins that are at least 30% identical, or at least 40% identical, preferably 50% identical, more preferably 60% identical, even more preferably 70% or even 80% identical, and most preferably 90% identical, at the amino acid level to the C. albicans CET1 protein. With respect to the C. albicans ABD1, highly related gene (homolog) is meant homologs encoding proteins that are at least 50% identical, preferably 60% identical, more preferably 70% identical, even more preferably 80% identical, and most preferably 90% identical, at the amino acid level. Percent similarity may be determined, for example, by comparing sequence information using the BLAST computer program, version 2.0, available on the World-Wide Web at http://www.ncbi.nlm.nih.gov. Typical parameters for determining the similarity of two sequences using BLAST 2.0 are a reward for match of 1, penalty for mismatch of −2, open gap and extension gap penalties of 5 and 2, respectively, a gap dropoff of 50, and a word size of 11. Highly related homologs (from Candida or other fungi) can encode proteins that are modulators of capping enzyme activities (for example, in a manner similar to the modulation of eIF4G by eIF4E-bp's). Modulators of enzyme activity will usually share a homologous protein domain. The invention also encompasses highly related genes (homologs) in other fungal species that preferably encode the corresponding TP'ase (in the case of CET1) or MT'ase (in the case of ABD1) capping enzymes. Other homologs of CET1 and/or ABD1 genes are those genes that encode proteins having 100% identity over 6 consecutive amino acids, and more preferably 8 amino acids, yet more preferably 15 amino acids, or even 20 amino acids. Production of the foregoing proteins and derivatives, e.g., by recombinant methods, is also provided.

The CET1 and ABD1 genes of the invention are preferably from species of fungal genus such as Candida, Aspergillus, Cryptococcus, Microsporum, Blastomyces, Pneumocystis, Histoplasma, Coccidioides, Mucor, Rhizopus, Trichosporon, Fusarium, Geotrichium, Pseudallescheria, Penicillium, Curvularia and Cunninghamella. In a preferred embodiment of the invention, the CET1 and ABD1 genes and proteins are from Candida spp. and particularly preferrably from C. albicans. Many Candida spp. are partial diploids, and there is also variance between different strains. As such, different strains will contain variants and allelic forms of the CET1 and ABD1 proteins, and polynucleotides encoding them are within the scope of the invention. Genes encoding CET1 and/or ABD1 proteins from other fungal species, and particularly Candida spp., can be cloned using labeled DNA probes made from nucleic acid fragments corresponding to any portion of the polynucleotides disclosed herein. More specifically, a library (either a genomic library or a cDNA derived library) from the fungal species or strain of interest is plated out and probed under appropriate conditions with labeled polynucleotides corresponding to portions of the ABD1 or CET1 genes disclosed herein. Methods of preparing and screening fungal libraries are well known to those of skill (see for example, the techniques described in Ausubel et al., 1989, Current Protocols in Molecular Biology, Greene Publishing Associates and Wiley Interscience, N.Y.).

The invention also relates to CET1 and/or ABD1 derivatives, truncations and analogs of the invention that are functionally active, i.e., they are capable of displaying one or more known functional activities associated with a full-length (wild-type) CET1 or ABD1 protein, and the nucleic acids encoding them.

The invention further relates to fragments (and derivatives and analogs thereof) of CET1 and/or ABD1 that comprise one or more domains of these proteins.

The invention also provides isolated or purified nucleic acids consisting of at least 8 nucleotides (i.e., a hybridizable portion) of a CET1 or an ABD1 sequence; in other embodiments, the nucleic acids consist of at least 25 (continuous) nucleotides, 50 nucleotides, 100 nucleotides, 150 nucleotides, or 200 nucleotides of a CET1 or an ABD1 sequence, or a full-length CET1 or ABD1 coding sequence. In another embodiment, the nucleic acids are smaller than 35, 200 or 500 nucleotides in length. Nucleic acids can be single or double stranded. The invention also relates to nucleic acids that selectively hybridize to or complementary to the foregoing sequences. In specific aspects, nucleic acids are provided that comprise a sequence complementary to at least 10, 25, 50, 100, or 200 nucleotides or the entire coding region of a CET1 or ABD1 coding sequence. Such nucleotides are useful for, inter alia, cloning naturally occurring CET1 or ABD1 genes and isolating CET1 or ABD1 homologs as described below.

For example, such nucleotides can be used as primers in a polymerase chain reaction (PCR) reaction to clone CET1 or ABD1 homologs from other species. PCR is used to amplify the desired sequence in a genomic or cDNA library, prior to selection. Oligonucleotide primers representing known ABD1 or CET1 sequences can be used as primers in PCR. In a preferred aspect, the oligonucleotide primers represent at least part of the ABD1 or CET1 conserved segments of strong homology between CET1 or ABD1 genes of different species. The synthetic oligonucleotides may be utilized as primers to amplify by PCR sequences from a source (RNA or DNA), preferably a cDNA library, of potential interest. PCR can be carried out, e.g., by use of a Perkin-Elmer Cetus thermal cycler and Taq polymerase (Gene Amp). The DNA being amplified can include mRNA or cDNA or genomic DNA from any eukaryotic species. One can choose to synthesize several different degenerate primers, for use in the PCR reactions. It is also possible to vary the stringency of hybridization conditions used in priming the PCR reactions, to allow for greater or lesser degrees of nucleotide sequence similarity between the known CET1 or ABD1 gene nucleotide sequence and the nucleic acid homolog being isolated. For cross species hybridization, low stringency conditions are preferred. For same species hybridization, moderately stringent conditions are preferred. After successful amplification of a segment of a CET1 or ABD1 homolog, that segment may be molecularly cloned and sequenced, and utilized as a probe to isolate a complete cDNA or genomic clone, as described below. This, in turn, will permit the determination of the gene's complete nucleotide sequence, the analysis of its expression, and the production of its protein product for functional analysis, as described infra. In this fashion, additional genes encoding CET1 or ABD1 proteins and CET1 or ABD1 analogs may be identified.

In a specific embodiment, a nucleic acid that is hybridizable to a CET1 or ABD1 nucleic acid (e.g., having sequence SEQ ID NO:1 or SEQ ID NO:3, or sequence that encodes SEQ ID NO:2 or SEQ ID NO:3) or its complement, or to a nucleic acid encoding a CET1 or ABD1 derivative, under conditions of low stringency is provided. By way of example and not limitation, procedures using such conditions of low stringency are as follows (see also Shilo and Weinberg, 1981, Proc. Natl. Acad. Sci. USA 78:6789-6792): Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 Ag/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

In another specific embodiment, a nucleic acid that is hybridizable to a CET1 or an ABD1 nucleic acid under conditions of moderate stringency is provided. For example, procedures using such conditions of moderate stringency are as follows: Filters containing DNA are pretreated for 6 h at 55° C. in a solution containing 6× SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 h at 55° C., and then washed twice for 30 minutes at 60° C. in a solution containing 1×SSC and 0.1% SDS. Filters are blotted dry and exposed for autoradiography. Other conditions of moderate stringency which may be used are well-known in the art. Washing of filters is done at 37° C. for 1 h in a solution containing 2X SSC, 0.1% SDS.

In another preferred embodiment of the invention, a nucleic acid that is hybridizable to a CET1 or ABD1 nucleic acid under conditions of high stringency is provided. By way of example and not limitation, procedures using such conditions of high stringency are as follows: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.lX SSC at 50° C. for 45 min before autoradiography. Other conditions of high stringency which may be used are well known in the art. The invention also encompasses the proteins and polypeptides encoded by these hybridizable nucleic acids described above.

5.2 Methods of Expression and Purification of Capping Enzymes

For many applications of the invention, purified capping enzymes, both fungal and metazoan, are advantageous. Methods of purifying capping enzymes from a wide variety of species are well known in the art and described in the literature (see, for example, the literature cited above in Section 2). Overproduction of capping enzymes from cloned expression constructs in genetically engineered hosts has been described for several metazoan and S. cerevisiae genes (again, see Section 2) and is also described herein below both generally and by way of working examples.

5.2.1 Expression of the CET1 or ABD1 Genes

The nucleotide sequence coding for a CET1 and/or ABD1 protein or a functionally active analog or fragment or other derivative thereof (see Section 5.1), can be inserted into an appropriate expression vector, i.e., a vector which contains the necessary elements for the transcription and translation of the inserted protein-coding sequence. The necessary transcriptional and translational signals can also be supplied by the native CET1 and/or ABD1 gene and/or its flanking regions. A variety of host-vector systems may be utilized to express the protein-coding sequence. These include but are not limited to microorganisms such as yeast containing yeast vectors, or bacteria transformed with bacteriophage, DNA, plasmid DNA, or cosmid DNA, insect cells and mammalian systems. The expression elements of vectors vary in their strengths and specificities. Depending on the host-vector system utilized, any one of a number of suitable transcription and translation elements may be used. In specific embodiments, the C. albicans CET1 or ABD1 gene is expressed, or a sequence encoding a functionally active portion of these proteins. In yet another embodiment, a fragment of the CET1 or ABD1 genes comprising a domain of one of these proteins is expressed.

Any of the methods previously described for the insertion of DNA fragments into a vector may be used to construct expression vectors containing a chimeric gene consisting of appropriate transcriptional/translational control signals and the protein coding sequences. These methods may include in vitro recombinant DNA and synthetic techniques and in vivo recombinants (genetic recombination). Expression of nucleic acid sequence encoding a CET1 or ABD1 protein or peptide fragment may be regulated by a second nucleic acid sequence so that the protein or peptide is expressed in a host transformed with the recombinant DNA molecule. Expression of a CET1 or ABD1 protein may be controlled by any promoter/enhancer element known in the art. Promoters which may be used to control CET1 or ABD1 expression include, but are not limited to, prokaryotic expression vectors such as the ~-lactamase promoter (Villa-Kamaroff, et al., 1978, Proc. Natl. Acad. Sci. U.S.A. 75:3727-3731), the tac promoter (DeBoer, et al., 1983, Proc. Natl. Acad. Sci. U.S.A. 80:21-25), T7 and T5 bacteriophage systems, the trp promoter; promoter elements from yeast or other fungi such as the Gal4 promoter, the ADH (alcohol dehydrogenase) promoter, PGK (phosphoglycerol kinase) promoter, and the alkaline phosphatase promoter; the tet inducible promoter (applicable to either bacterial or eukaryotic systems); and CMV promoter for mammalian systems.

In addition, a host cell strain may be chosen that modulates the expression of the inserted sequences, or modifies and processes the gene product in the specific fashion desired. Expression from certain promoters can be elevated in the presence of certain inducers; thus, expression of the genetically engineered protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system can be used to produce an unglycosylated core protein product. Expression in yeast can be used to produce a glycosylated product.

In other specific embodiments, the CET1 or ABD1 protein, fragment, analog, or derivative may be expressed as a fusion, or chimeric protein product (comprising the protein, fragment, analog, or derivative joined via a peptide bond to a heterologous protein sequence (of a different protein)). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

In a specific embodiment, an expression construct is made by subcloning a CET1 or ABD1 coding sequence into the EcoRI restriction site of each of the three PGEX vectors (Glutathione S-Transferase (GST) expression vectors; Smith and Johnson, 1988, Gene 7:31-40). This procedure allows for the expression of the protein product from the subclone in the correct reading frame. The GST tag allows for the easy identification and purification of the resulting fusion protein. Other widely used protein tags are the His-tag or the Flag peptide (Hopp et al., 1988, Bio/Technol. 6:1204).

Both cDNA and genomic sequences can be cloned and expressed. Furthermore, using the well-known degeneracy of the genetic code, the codon usage of the nucleic acids of the 30 invention can be tailored for optimal expression in the host cell chosen for expression. Additionally, for expression of Candida genes in other organisms, codon usage for particular amino acids should be altered (e.g., CUG encodes serine in Candida, but encodes leucine in other species).

In specific embodiments of the invention, described below by way of example, fungal capping enzymes are expressed in E. coli from an IPTG-inducible expression construct.

5.2.2 Identification and Purification of the CET1 or ABD1 Gene Products

In particular aspects, the invention provides amino acid sequences of CET1 and ABD1 proteins, preferably C. albicans CET1 and ABD1 proteins, and fragments and derivatives thereof which comprise an antigenic determinant (i.e., can be recognized by an antibody) or which are otherwise functionally active, as well as nucleic acid sequences encoding the foregoing. “Functionally active” CET1 or ABD1 material as used herein refers to that material displaying one or more known functional activities associated with a full-length (wild-type) CET1 or ABD1 protein, e.g., enzymatic activity, binding to an RNA substrate or other enzyme, antigenicity (binding to an anti-CET1 or ABD1 antibody), immunogenicity, etc.

In specific embodiments, the invention provides fragments of a CET1 or ABD1 protein consisting of at least 6 amino acids, 10 amino acids, 50 amino acids, or of at least 75 amino acids. Such fragments are useful as antigenic peptides. In other embodiments, the proteins comprise or consist essentially of specific domains of ABD1 or CET1, or any combination of such domains. With respect to CET1, the enzymatic activity and protein interaction domain resides in about amino acid residues 173 to 520 of SEQ ID NO:2. In particular, the sequence PIWAQXWXP from amino acid residues 206 to 214 of SEQ ID NO:2 can define a GT'ase interaction domain of the CET1 protein. Additionally, three triphosphatase motifs occur from amino acids 283-297, 438-451, and 464-476 of SEQ ID NO:2; each of these domains is likely involved in the catalytic site of this enzyme. With respect to ABD1, amino acid residues 158 to 474 of SEQ ID NO:4 contain the core domain required for enzymatic activity, amino acid residues 138 to 474 of SEQ ID NO:4 contain the core domain required for fungal cell viability, amino acid residues 138 to 158 of SEQ ID NO:4 encompasses an ABD1 domain involved in interacting with other cellular components (e.g. triphosphatase and/or guanylyltransferase and/or RNA polymerase II), and amino acid residues 203 to 217 of SEQ ID NO:4 contain a motif involved in binding the AdoMet substrate. Fragments, or proteins comprising fragments, lacking some or all of the foregoing regions of a CET1 or ABD1 protein are also provided. As noted above, nucleic acids encoding the foregoing are provided.

Once a recombinant polynucleotide that expresses the CET1 or ABD1 gene sequence is identified, the gene product can be analyzed. This is achieved by assays based on the physical or functional properties of the product (e.g. enzymatic activity as described below in the examples), including radioactive labeling of the product followed by analysis by gel electrophoresis, TLC chromatography, immunoassay, etc.

The CET1 and ABD1 proteins and polypeptides of the invention can be isolated and purified by standard methods including chromatography (e.g., ion exchange, affinity, and sizing column chromatography), centrifugation, differential solubility, or by any other standard technique for the purification of proteins. The functional properties may be evaluated using any suitable assay (e.g., see Examples).

Alternatively, once the CET1 or ABD1 protein produced by a recombinant is identified, the amino acid sequence of the protein can be deduced from the nucleotide sequence of the chimeric gene contained in the recombinant. As a result, the protein can be synthesized by standard chemical methods known in the art (e.g., see Hunkapiller, M., et al., 1984, Nature 310:105-111).

In another alternate embodiment, native CET1 or ADD1 proteins can be purified from natural sources, by standard methods such as those described herein and in the literature (e.g., differential solubility, chromatography, and/or immunoaffinity purification).

In a specific embodiment of the present invention, such CET1 or ABD1 proteins, whether produced by recombinant DNA techniques or by chemical synthetic methods or by purification of native proteins, include but are not limited to those containing, as a primary amino acid sequence, all or part of the amino acid sequence substantially as depicted in SEQ ID NOs:2 and 4, as well as fragments and other derivatives, and analogs thereof, including proteins homologous thereto.

5.3 Generation of Antibodies to CET1 and ABD1 Polypeptides

According to the invention, CET1 and/or ABD1 proteins, their fragments or other derivatives, or analogs thereof, may be used as an immunogen to generate antibodies that immunospecifically bind such an immunogen. Such antibodies include but are not limited to polyclonal, monoclonal, chimeric, single chain, Fab fragments, and an Fab expression library. In one embodiment, antibodies to a domain of a CET1 and/or ABD1 protein are produced. In a specific embodiment, fragments of a CET1 and/or ABD1 protein identified as hydrophilic are used as immunogens for antibody production.

Various procedures known in the art may be used for the production of polyclonal antibodies to a CET1 or ABD1 protein or derivative or analog. In a particular embodiment, rabbit polyclonal antibodies to an epitope of a CET1 or ABD1 protein encoded by a sequence of SEQ ID NOs:2 or 4, or a subsequence thereof, can be obtained. For the production of antibody, various host animals can be immunized by injection with the native protein, or a synthetic version, or derivative (e.g., fragment) thereof, including but not limited to rabbits, mice, rats, etc. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed toward a CET1 or ABD1 protein sequence or analog thereof, any technique that provides for the production of antibody molecules by continuous cell lines in culture may be used. For example, the hybridoma technique originally developed by Kohler and Milstein (1975, Nature 256:495-497), as well as 15 the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) 30 by splicing the genes from a mouse antibody molecule specific for the target protein together with genes from a human antibody molecule of appropriate biological activity can be used; such antibodies are within the scope of this invention.

According to the invention, techniques described for the production of single chain antibodies (U.S. Pat. No. 4,946,778) can be adapted to produce CET1 or ABD1-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for CET1 or ABD1 proteins, derivatives, or analogs.

Antibody fragments that contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). For example, to select antibodies that recognize a specific domain of a CET1 or ABD1 protein, one may assay generated hybridomas for a product that binds to a CET1 or ABD1 fragment containing such domain. For example, one can select an antibody that specifically binds a first CET1 homolog but which does not specifically bind a different CET1 homolog, on the basis of positive binding to the first CET1 homolog and a lack of binding to the second CET1 homolog.

Antibodies specific to a domain of a CET1 or an ABD1 protein are also provided.

The foregoing antibodies can be used in methods known in the art relating to the localization and activity of the CET1 and/or ABD1 protein sequences of the invention, e.g., for imaging these proteins, measuring levels thereof in appropriate physiological samples, in screening assays, etc.

5.4 Uses of Fungal Capping Enzymes For Drug Discovery

5.4.1 Screening Assays

Another aspect of the invention is to provide assays useful for identifying compounds that interfere with fungal capping processes. In a first level screen, assays are provided for determining if a compound of interest can bind to CET1 or ABD1 so as to interfere with activity of the protein. Assays are described below that are designed to identify compounds that interact with (e.g., bind to) CET1 or ABD1, and compounds that interfere with the interaction of CET1 or ABD1 with other intracellular proteins or with mRNA, including but not limited to compounds that interfere with the interaction of any two or more of the following proteins: CET1, ABD1, CGT1 and RNA polymerase II. Assays may additionally be utilized which identify compounds that modulate the activity of the CET1 or ABD1 gene (i.e., modulate the level of CET1 or ABD1 gene expression) or that bind to CET1 or ABD1 gene regulatory sequences (e.g., promoter sequences) and which may modulate CET1 or ABD1 gene expression. See e.g., Platt, K.A., 1994, J. Biol. Chem. 269:28558-28562. In a second level type of screen, compounds are assayed for their ability to inhibit any one or all steps of the fungal capping reaction. Such assays are described below both generally and by way of specific, non-limiting examples.

The compounds that may be screened in accordance with the invention include but are not limited to peptides, antibodies and fragments thereof, prostaglandins, lipids and other organic compounds (e.g., terpines, peptidomimetics), as well as inorganic compounds. Peptides can include, but are not limited to, soluble peptides, members of random peptide libraries (see, e.g., Lam, K. S. et al., 1991, Nature 354:8284; Houghten, R. et al., 1991, Nature 354:84-86), and combinatorial chemistry-derived molecular library peptides made of D- and/or L- configuration amino acids, phosphopeptides (including, but not limited to members of random or partially degenerate, directed phosphopeptide libraries; see, e.g., Songyang, Z. et al., 1993, Cell 72:767-778). Antibodies can be polyclonal, monoclonal, humanized, anti-idiotypic, chimeric or single chain antibodies, FAb, F(ab′ )₂ and FAb expression library fragments, and epitope-binding fragments thereof).

Other compounds that can be screened in accordance with the invention include but are not limited to small organic molecules that are able to gain entry into a cell and affect the expression of the CET1 or ABD1 gene (e.g., by interacting with the regulatory region or transcription factors involved -in gene expression); or such compounds that affect the activity of CET1 or ABDD (e.g., by inhibiting or enhancing the binding of CET1 or ABD1 to mRNA other substrate).

A number of compound libraries are commercially available from companies such as Pharmacopeia, ArQule, Enzymed, Sigma, Aldrich, Maybridge, Trega and PanLabs, to name just a few sources. One can also screen libraries of known compounds, including natural products or synthetic chemicals, and biologically active materials, including proteins and natural product extracts, for compounds that are inhibitors of fungal capping reactions.

Additionally, once a compound that affects a binding interaction is identified, molecular modeling techniques can be used to design variants of the compound that are more effective. Examples of molecular modeling systems are the CHARM and QUANTA programs (Polygen Corporation, Waltham, Mass.). CHARM performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins, such as Rotivinen et al., 1988, Acta Pharmaceutical Fennica 97:159-166; Ripka, New Scientist 54-57 (Jun. 16, 1988); McKinaly and Rossmann, 1989, Annu. Rev. Pharmacol. Toxiciol. 29:111-122; Perry and Davies, OSAR: Quantitative Structure-Activity Relationships in Drug Design pp. 189-193 (Alan R. Liss, Inc. 1989); Lewis and Dean, 1989, Proc. R. Soc. Lond. 236:125-140 and 141-162; and, Askew et al., 1989, J. Am. Chem. Soc. 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to any identified region.

Compounds identified via assays such as those described herein may be useful, for example, in treating conditions associated with fungal infections. Assays for testing the effectiveness of compounds are discussed below.

5.4.1.1 Binding Assay Formats

The principle of the assays used to identify compounds that bind to the CET1 or ABD1 involves preparing a reaction mixture of the CET1 or ABD1 protein and the test compound under conditions and for a time sufficient to allow the two components to interact and bind, thus forming a complex which can be removed and/or detected in the reaction mixture. The CET1 or ABD1 species used can vary depending upon the goal of the screening assay. For example, where compounds that interfere with a particular binding domain are sought, the full length CET1 or ABD1 containing that binding domain, the binding domain itself, or a fusion protein containing CET1 or ABD1 fused to a protein or polypeptide that affords advantages in the assay system (e.g., labeling, isolation of the resulting complex, etc.) can be utilized. The peptides derived from the capping enzymes for use in this technique should comprise at least 6 consecutive amino acids, preferably 10 consecutive amino acids, more preferably 20 consecutive amino acids, even more preferably 30 or even 50 consecutive amino acids, or more, of the amino acid sequences provided herein.

The screening assays can be conducted in a variety of ways. For example, one method to conduct such an assay would involve anchoring the CET1 or ABD1 protein, polypeptide, peptide or fusion protein or the test substance onto a solid phase and detecting CET1 or ABDl/test compound complexes anchored on the solid phase at the end of the reaction. In one embodiment of such a method, the CET1 or ABD1 reactant may be anchored onto a solid surface, and the test compound, which is not anchored, may be labeled, either directly or indirectly. Alternatively, the test compound can be anchored to a solid support. Any of a variety of suitable labeling systems can be used including but not limited to radioisotopes such as ¹²⁵I and ³²P, enzyme labelling systems that generate a detectable calorimetric signal or light when exposed to a substrate, and fluorescent labels. In another embodiment of the method, a CET1 or ABD1 protein anchored on the solid phase is complexed with labeled antibody. Then, a test compound could be assayed for its ability to disrupt the association of the CET1 or ABD1/antibody complex.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Another solid support system particularly advantageous for screening is the BIAcore 2000™ system, available commercially from BIAcore, Inc. (Piscataway, N.J.). The BIAcoreTM instrument (http://www.biacore.com) uses the optical phenomenon of surface plasmon resonance (SPR) to monitor biospecific interactions in real-time. The SPR effect is essentially an evanescent electrical field that is affected by local changes in refractive index at a metal-liquid interface. A sensor chip made up of a sandwich of gold film between glass and a carboxymethyl dextran matrix to which the ligand or protein to be assayed is chemically linked. This sensor chip is mounted on a fluidics cartridge which forms flow cells through which analyte compounds can be injected. Ligand-analyte interactions on the sensor chip are detected as changes in the angle of a beam of polarized light reflected from the chip surface. Binding of any mass to the chip affects SPR in the gold/dextran layer. This change in the electrical field in the gold layer interacts with the reflected light beam and alters the angle of reflection proportional to the amount of mass bound. Reflected light is detected on a diode array and translated to a binding signal expressed as response units (RU). As the response is directly proportional to the mass bound, kinetic and equilibrium constants for protein-protein interactions can be measured.

Alternatively, a reaction can be conducted in a liquid phase, the reaction products separated from unreacted components, and complexes detected; e.g., using an immobilized antibody specific for CET1 or ABD1 protein, polypeptide, peptide or fusion protein, or the test compound to anchor any complexes formed in solution, and a labeled antibody specific for the other component of the possible complex to detect anchored complexes.

5.4.1.2 Assay Formats for Compounds That Disrupt Binding Partners

The macromolecules that interact with the CET1 or ABD1 protein are referred to, for purposes of this discussion, as “binding partners”. The binding partners of interest here are the substrates (such as mRNA), or other cellular factors, that bind to CET1 or ABD1. Other cellular factors that bind to the CET1 TP'ase protein include but are not limited to the CGT1 (GT'ase) protein, RNA polymerase II and RNA. Intracellular binding partner proteins for ABD1 include, for example, RNA polymerase II and RNA. Therefore, it is desirable to identify compounds that interfere with or disrupt the interaction of such binding partners with CET1 or ABD1 which may be useful in regulating the activity of CET1 or ABD1 and thus mRNA capping reactions.

The basic principle of the assay systems used to identify compounds that interfere with the interaction between the CET1 or ABD1 protein and its binding partner or partners involves preparing a reaction mixture containing CET1 or ABD1 protein, polypeptide, peptide or fusion protein as described above, and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the CET1 or ABD1 moiety and its binding partner. Control reaction mixtures are incubated without the test compound or with a placebo. The formation of any complexes between the CET1 or ABD1 moiety and the binding partner is then detected. The formation of a complex in the control reaction, but not in the reaction mixture containing the test compound, indicates that the compound interferes with the interaction of CET1 or ABD1 and the interactive binding partner.

The assay for compounds that interfere with the interaction of CET1 or ABD1 and binding partners can be conducted in a heterogeneous or homogeneous format. Heterogeneous assays involve anchoring either CET1 or ABD1 moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with CET1 or ABD1 moiety and interactive binding partner. Alternatively, test compounds that disrupt preformed complexes, e.g. compounds with higher binding constants that displace one of the components from the complex, can be tested by adding the test compound to the reaction mixture after complexes have been formed. In many cases, the various formats are essentially modifications of the binding assays described above.

In a particular embodiment, a CET1 or ABD1 fusion protein can be prepared for immobilization. For example, CET1 or ABD1 or a peptide fragment, e.g., corresponding to a fragment of CET1 containing the CGT1 protein interaction domain, can be fused to a glutathione-S-transferase (GST) gene using a fusion vector, such as pGEX-5X-1, in such a manner that its binding activity is maintained in the resulting fusion protein. The interactive binding partner can be labeled with radioactive isotope, for example, by methods routinely practiced in the art. In a heterogeneous assay, e.g., the GST-CET1 or GST-ABD1 fusion protein can be anchored to glutathione-agarose beads. The interactive binding partner can then be added in the presence or absence of the test compound in a manner that allows interaction and binding to occur. At the end of the reaction period, unbound material can be washed away. The interaction between the CET1 or ABD1 gene product and the labeled interactive binding partner can be detected by measuring the amount of radioactivity that remains associated with the glutathione-agarose beads. A successful inhibition of the interaction by the test compound will result in a decrease in measured radioactivity.

Alternatively, the GST-CET1 or GST-ABD1 fusion protein and the labeled interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of CET1 or ABD1/binding partner interaction can be detected by measuring the radioactivity associated with the beads.

In another embodiment of the invention, these same techniques can be employed using peptide fragments that correspond to the binding domains of CET1 or ABD1, in place of the full length proteins. Any number of methods routinely practiced in the art can be used to identify and isolate the binding sites. These methods include, but are not limited to, mutagenesis of the gene encoding the protein and screening for disruption of binding in a co-immunoprecipitation assay. Sequence analysis of the gene encoding the protein will reveal the mutations that correspond to the region of the protein involved in interactive binding.

In still another aspect of the invention, screens for compounds that interfere with binding can be performed by assaying for disruption of an energy transfer event between the two binding partners. Specifically, one binding partner is labeled with a moiety that, when brought into close proximity with a second moiety labeling the second binding partner, results in a transfer of energy between the two moieties on the two binding partners. This transfer of energy can be detected by a change in wavelength of emitted light. An example is time-resolved fluorescence assay (HTRF) commercially available from Packard Instrument Co., Meriden, Conn.

5.4.1.3 In vivo Binding Assays

Other aspects of the invention are in vivo screens for CET1 and/or ABD1 binding partners, and for agents that disrupt interaction of CET1 or ABD1 with their binding partners. One method that detects protein interactions in vivo, the two-hybrid system, is well known to those of skill in the art and is commercially available from Clontech (Palo Alto, Calif.).

Briefly, when utilizing such a system, plasmids are constructed that encode two hybrid proteins: one plasmid consists of nucleotides encoding the DNA-binding domain of a transcription activator protein fused to a capping enzyme-encoding nucleotide sequence, and the other plasmid consists of nucleotides encoding the transcription activator protein's activation domain fused to a cDNA encoding an unknown protein which has been recombined into this plasmid as part of a cDNA library (when searching for binding partners) or a known protein. The cDNA library is prepared from a cell known to contain proteins that interact with the capping enzyme protein, such as other fungal cells. The DNA-binding domain fusion plasmid and the cDNA library are transformed into a strain of the yeast Saccharomyces cerevisiae that contains a reporter gene (e.g., HIS or lacZ) whose regulatory region contains the transcription activator's binding site. Either hybrid protein alone cannot activate transcription of the reporter gene; the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which in turn is detected by an assay for the reporter gene product.

Additionally, yeast cells containing interacting two-hybrid binding partners may be used as test organism for compounds that interfere with the interaction. For example, yeast two-hybrid screen can be used to screen for compounds that affect the interaction between the CET1 TP'ase and a CGT1 GT'ase.

5.4.2 Capping Enzyme Activity Assays

Assays for each step of the RNA capping process are provided. FIG. 2 illustrates the three fundamental steps of mRNA capping. Such assays are useful in monitoring enzyme activity during purification, as well as in screens of compounds that inhibit one or more fungal capping activity.

Briefly, fungal triphosphatase enzymes catalyze the hydrolysis of the γ-P of pppRNA to liberate free inorganic phosphate (see FIG. 2, step (1)). Assays for use in the invention monitor the release of inorganic phosphate from a 5′ triphosphate end labeled substrate RNA molecule. The liberated phosphate may be detectably labeled, or may be monitored by indirect techniques such as a phosphate assay. Various examples of triphosphatase assays for use in the invention are provided below by way of exemplary embodiments.

Guanylyltransferase catalyzes a two-step reaction (see FIG. 2). For monitoring the first step of this reaction, the formation of an enzyme-GMP covalent intermediate is assayed. Such assays are described in the literature (see Yue et al., 1997, Proc. Natl. Acad. Sci. USA, 94:12898-12903; Shibagaki et al., 1992, J. Biol. Chem., 267:9521-9528; Yamada-Okabe et al., 1996, supra; Ho et al., 1998, J. Biol. Chem., 273:9577-9585; Itoh et al., 1984, J. Biol. Chem., 259:13923-13929). The second step of the reaction is assayed by monitoring either the release of pyrophosphate (PPi) (either labeled or unlabeled), or the generation of the GpppNpN(pN)ₙ product. The GpppNpN(pN)ₙ product can be easily identified by, for example, TLC. Examples of assays for both parts of the guanylyltransferase reaction are exemplified in detail below.

Assays to measure mRNA methyltransferase rely upon the detection, and optionally quantitation, of the transfer of a methyl group to guanylylated RNA (GpppNpN(pN),. A thin-layer chromatography (TLC) assay has been described (see Mao et al., 1995, Mol. Cell. Biol., 15:4167-4174; Ping-Wang and Shuman, 1997, J. Biol. Chem., 272:14683-14689) which relies upon separation of a radiolabeled substrate and its methylated product. Modifications of this method are provided which make use of a ³H-labeled substrate. Both types of assays are described in detail below by way of working examples.

Alternatively, since the methyltransferase step is the final capping step, and a cap structure is necessary for efficient translation, assays for detection can make use of a linked in vitro translation step. Such IVT assays conveniently produce a detectable product such as luciferase and green fluorescent protein or radiolabeled protein.

Each or all of the enzymatic steps in fungal capping are amenable to high throughput assays for candidate inhibitors. High through-put screens are well known in the art and can be performed in any of a number of formats. For example, filter assays, scintillation proximity technology, spectroscopic assays, light-based luciferase assays and HTRF energy transfer assays (Packard Instrument Company, Meriden, Conn.; see also U.S. Pat. Nos. 5,527,684 and 5,512,493) are useful formats. Laboratory automation, including robotics technology, can vastly decrease the time necessary to screen large numbers of compounds and is commercially available from, for example, Tecan, Scitec, Rosys, Mitsubishi, CRS Robotics, Fanuk, and Beckman-Coulter Sagian, to name just a few companies. After candidate inhibitors are identified (or concurrently with their identification), secondary screens are performed in parallel with mammalian capping enzymes in order to find agents selective for inhibition of fungal capping enzymes.

5.4.3 Fungal Capping Inhibitors

Inhibitory compounds identified in the foregoing screening assays which may be used in accordance with the invention may include but are not limited to small organic molecules, peptides and antibodies. Additionally, antisense compounds that are specifically targetted to the gene product of fungal capping enzyme genes can also be used to inhibit fungal capping.

For example, peptides having an amino acid sequence corresponding to the domain of the CET1 protein that binds to the CGT1 protein can be used to compete with the native CGT1 protein and, therefore, can be useful as inhibitors in accordance with the invention. Similarly, peptides having an amino acid sequence corresponding to the domain of the CGT1 protein that binds to the CET1 protein may be used. Such peptides may be synthesized chemically or produced via recombinant DNA technology using methods well known in the art (e.g., see Creighton, 1983, supra; and Sambrook, et al., 1989, supra). Lipofectin or liposomes can be used to deliver the peptides to cells.

Alternatively, antibodies that are both specific for the binding domains or active sites of either CET1 or ABD1, or other capping enzymes, and interfere with their interaction or activity may be used. Such antibodies may be generated using standard techniques described in Section 5.3, supra, against the proteins themselves or against peptides corresponding to the binding domains of the proteins. Such antibodies include but are not limited to polyclonal, monoclonal, Fab fragments, single chain antibodies, chimeric antibodies, etc. Where whole antibodies are used, internalizing antibodies are preferred. However, lipofectin may be used to deliver the antibody or a fragment of the Fab region which binds to the fungal cell protein epitope into cells. Where fragments of the antibody are used, the smallest inhibitory fragment that binds to the target protein's binding domain is preferred.

In another embodiment, capping enzyme function is I inhibited by use of antisense nucleic acids specific to the capping enzyme genes. The present invention provides the therapeutic or prophylactic use of nucleic acids of at least six nucleotides that are antisense to a gene or cDNA encoding a capping enzyme gene or a portion thereof. An “antisense” nucleic acid as used herein refers to a nucleic acid capable of hybridizing to a portion of an RNA (preferably mRNA) by virtue of some sequence complementarity. The antisense nucleic acid may be complementary to a coding and/or noncoding region of an mRNA. Preferrably, the antisense nucleic acids are complementary to the CET1 and ABD1 genes of the invention, but the invention also encompasses the use of antisense nucleic acids complementary to any other fungal capping enzymes. Such antisense nucleic acids have utility as therapeutics that inhibit capping enzyme function, and can be used in the treatment of fungal infections as described in Section 5.5.

The antisense nucleic acids of the invention can be oligonucleotides that are double-stranded or single-stranded, RNA or DNA or a modification or derivative thereof, which can be directly administered to a fungal cell, or which can be produced intracellularly by transcription of exogenous, introduced sequences.

The invention further provides pharmaceutical compositions comprising an effective amount of the antisense nucleic acids of the invention in a pharmaceutically acceptable carrier, as described infra.

For convenience, the antisense nucleic acids and their uses are described in detail below with reference to CET1 and ABD1 antisense nucleic acids. However, the invention encompasses antisense nucleic acids complementary to other fungal capping enzyme genes.

The CET1 and ABD1 antisense nucleic acids are of at least six nucleotides and are preferably oligonucleotides (ranging from 6 to about 50 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO 88/09810, published Dec. 15, 1988), hybridization-triggered cleavage agents (see, e.g., Krol et al., 1988, BioTechniques 6:958-976) or intercalating agents (see, e.g., Zon, 1988, Pharm. Res. 5:539-549) and conjugates are those that will target the oligonucleotide to fungal cells such as antibodies to fungal determinants.

In a preferred aspect of the invention, a CET1 and/or ABD1 antisense oligonucleotide is provided, preferably of single-stranded DNA. In a most preferred aspect, such an oligonucleotide comprises a sequence antisense to the sequence encoding the methione initiator codon and the N terminus of the polypeptide. The oligonucleotide may be modified at any position on its structure with substituents generally known in the art.

The CET1 and ABD1 antisense oligonucleotides can comprise at least one modified base moiety selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including but not limited to arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

In a specific embodiment, the CET1 and/or ABD1 antisense oligonucleotide comprises catalytic RNA, or a ribozyme (see, e.g., PCT International Publication WO 90/11364, published Oct. 4, 1990; Sarver et al., 1990, Science 247:1222-1225). In another embodiment, the oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

The antisense nucleic acids of the invention comprise a sequence complementary to at least a portion of an RNA transcript of a CET1 or ABD1 gene, preferably a C. albicans gene. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded GENE antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a CET1 or ABD1 RNA it may contain and still form a stable duplex (or triplex, as the case may be). One skilled in the art can ascertain a tolerable degree of mismatch by use of standard procedures to determine the melting point of the hybridized complex.

Pharmaceutical compositions of the invention (see Section 5.5), comprising an effective amount of a CET1 or ABD1 antisense nucleic acid in a pharmaceutically acceptable carrier, can be administered to a patient having a fungal infection. The amount of CET1 or ABD1 antisense nucleic acid effective in the treatment of a particular disorder or condition will depend on the nature of the disorder or condition, and can be determined by standard clinical techniques In a specific embodiment, pharmaceutical compositions comprising CET1 or ABD1 antisense nucleic acids are administered via liposomes, microparticles, or microcapsules. In various embodiments of the invention, it may be useful to use such compositions to achieve sustained release of the CET1 or ABD1 antisense nucleic acids. In a specific embodiment, it may be desirable to utilize liposomes targeted via antibodies to specific fungal antigens (Leonetti et al., 1990, Proc. Natl. Acad. Sci. U.S.A. 87:2448-2451; Renneisen et al., 1990, J. Biol. Chem. 265:16337-16342).

5.4.4 Assays For Inhibition of Fungal Infections

Compounds, including but not limited to binding compounds and enzymatic inhibitors identified via assay techniques such as those described above and in the Examples, can be tested for the ability to ameliorate conditions associated with fungal infections. By inhibiting fungal mRNA capping through the CET1 or ABD1 proteins, fungal growth can be arrested or eliminated. The assays described above can identify compounds that affect CET1 or ABD1 activity (e.g., compounds that bind to CET1 or ABD1, inhibit binding of the natural ligands, or activate binding of the natural ligands, and compounds that bind to a natural ligand of CET1 or ABD1 and neutralize the ligand activity, and compounds that inhibit enzymatic activity); or compounds that affect CET1 or ABD1 gene activity (by affecting CET1 or ABD1 gene expression, including molecules, e.g., proteins or small organic molecules, that affect or interfere with CET1 or ABD1 transcript stability). Such compounds can be used as part of a therapeutic method for the treatment of fungal infections.

The invention encompasses cell-based and animal model-based assays for the identification of compounds exhibiting such an ability to ameliorate fungal infections. These assay systems can also be used as the standard to assay for purity and potency of the compounds, including recombinantly or synthetically produced CET1 or ABD1 mutants.

Such cell-based systems can include, for example, fungal cells, mammalian cell lines maintained in vitro and mammalian cell/fungal co-cultures. Any kind of mammalian cell that can be grown in culture or any fungal cell can be used in cell based assay.

In one assay, fungal cells may be exposed to a test compound, and expression of the CET1 or ABD1 gene, e.g., by assaying cell lysates for CET1 or ABD1 mRNA transcripts (e.g., by Northern analysis) or for CET1 or ABD1 protein expressed in the cell is performed; compounds that regulate or modulate expression of the CET1 or ABD1 gene are valuable candidates as therapeutics. Or, more simply, fungal growth and viability is assayed after exposure to a test compound thought to inhibit capping activity. Similarly, the effect of a test compound on mammalian cell growth and viablitity may be assayed.

In another embodiment, compounds are tested for their differential effect on fungal cells genetically engineered to express either fungal capping enzymes or human capping enzymes. For example, various strains of S. cerevisiae are constructed in which the genes encoding endogenous capping enzymes are disabled, and capping activity is rescued by replacement with any combination of Candida capping enzymes or human capping enzymes.

In yet another embodiment utilizing such cell systems, mammalian cell/fungal co-cultures may be exposed to a compound suspected of inhibiting fungal capping activity, at a sufficient concentration and for a time sufficient to elicit such an effect in the exposed cells. After exposure, the cell co-cultures can be assayed to measure alterations in the ratio of mammalian to fungal cells, or differential survival of mammalian and fungal cells.

In addition, animal-based systems, which may include, for example, rats, mice, chicken, cows, monkeys, rabbits, etc., may be used to identify compounds capable of affecting fungal capping and, hence, fungal growth in vivo. Such animal models may be used as test systems for the identification of drugs, pharmaceuticals, therapies and interventions effective in treating such disorders in humans.

As an example, animal models of fungal infections may be exposed to a compound suspected of exhibiting an ability to interfere with the activity of CET1 or ABD1, and hence, fungal capping, at a sufficient concentration and for a time sufficient to elicit an amelioration of symptoms of fungal infection in the exposed animals. The response of the animals to the exposure may be monitored by assessing the reversal of disorders associated with fungal infection. With regard to intervention, any treatments that reverse any aspect of symptoms associated with fungal infections should be considered as candidates for human disorder therapeutics. Dosages of test agents may be determined by deriving dose-response curves, as discussed below.

5.5 Pharmaceutical Preparations and Administration

Polynucleotides encoding CET1 or ABD1, and derivatives thereof, and the compounds that are determined to affect CET1 or ABD1 gene expression or activity, or the interaction of these proteins with other fungal proteins, can be administered to a patient at therapeutically effective doses to treat or ameliorate diseases related to fungal infections. Such diseases include but are not limited to thrush, esophagitis, urinary tract infections, cutaneous or ocular lesions, meningitis, endocarditis, nosocomial infections, cryptococcal meningitis and aspergillosis. A therapeutically effective dose refers to that amount of the compound sufficient to result in amelioration of symptoms of fungal infection, including but not limited to rashes, skin eruptions, tissue degeneration, itching, pain, shortness of breath and decreased longevity.

When compounds identified in screening assays are to be delivered to a subject, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC., (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

5.6 Kits and Commercial Applications

The present invention also encompasses commercial kits comprising the novel fungal capping enzymes of the invention. In particular embodiments, the invention encompasses kits containing the C. albicans CET1 and/or ABD1 proteins and polypeptides described herein. The kits may also optionally contain one or more of the following components: a CGT1 (GT'ase) enzyme, directions for use; a buffer or buffer concentrate optimized for capping enzyme activity; substrates such as a control mRNA template; GTP (labeled or unlabeled), and/or AdoMet (again, optionally labeled). Yet another embodiment of the kits of the invention can also contain an inhibitor of mammalian TP'ase (e.g., a tyrosine phosphatase inhibitor) and appropriate buffers, etc.

Other commercial kits within the scope of the invention are diagnostic kits for the presence of fungal infections. Such kits can contain an antibody (monoclonal or polyclonal) or antibodies specific to an epitope on the fungal capping enzyme polypeptide of the invention. The antibody can be labeled directly, or the kit can contain a secondary label (e.g., an enzyme-linked second antibody). The kit can also contain appropriate buffers, control antibodies, and directions for use. Yet another diagnostic kit can contain a polynucleotide or polynucleotides useful for identifying the presence of fungal DNA or RNA (e.g., such as by the PCR reaction). Diagnostic kits are valuable for both clinical and research applications.

### 6. EXAMPLE

C. albican Guanylyltransferase and S. cerevisiae Methyltransferase Overexpression Plasmids

In order to guarantee sufficient quantities of the enzymes required for the development of a high-throughput screening assay, the relevant genes were obtained and cloned into isopropyl-D-thiogalactoside (IPTG)-inducible protein overexpression plasmids permitting production of the capping enzymes in Escherichia coli.

The C. albicans CGT1 and the S. cerevisiae ABD1 genes encoding the GT'ase and MT'ase, respectively, have been identified and cloned (Yamada-Okabe et al., 1996, supra; Mao et al., 1995, supra). The S. cerevisiae ABD1 gene was obtained as a phage lambda genomic clone (Clone # 70214) from the American Type Culture Collection (ATCC, Rockville, Md.). The C. albicans CGTi gene was obtained by polymerase chain reaction (PCR) amplification using C. albicans genomic DNA and specific primers based on the published sequence.

The CGT1 gene was subcloned into the E. coli protein overexpression vector pETIIc (Novagen, Milwaulki, Wis.) using PCR. This plasmid contains the T7 promoter and the rrnB terminator. This plasmid directed the expression of the GT'ase protein with unmodified N— and C-termini. However, due to the fact that C. albicans uses a non-canonical CUG serine codon, two serine residues (Ser-565 and Ser-595) of the native C. albicans enzyme were mutagenized to the universal serine codon (ACG) using oligonucleotide directed PCR mutagenesis. Confirmation of the DNA sequence of the entire gene was done by the dideoxy chain termination method.

The S. cerevisiae ABD1 gene encoding the MT'ase was subcloned into the protein expression vector pQE30 (Qiagen). This plasmid contains the phage T5 promoter, an efficient Shine-Dalgarno sequence, and a 12 amino acid N-terminal fusion sequence containing 6 histidine residues. This construct directed the expression of the S. cerevisiae MT'ase protein as an N-terminal hexa-histidine fusion protein. Confirmation of the DNA sequence of the entire gene was done by the dideoxy chain termination method.

### 7. EXAMPLE

Purification and Assay of C. albicans Guanylyltransferase

The E. coli strain containing the CGT1 overexpression plasmid was grown at 37° C. in LB medium supplemented with ampicillin (100 μg/ml). Cultures were grown to an absorbance (at 600 nm) of 0.4, then induced to overexpress the cloned protein by the addition of IPTG to 0.5 mM. Growth was continued for an additional 3 hours, after which the cells were harvested by centrifugation and resuspended to 20% (w/v) in Buffer B (50 mM Tris-Cl pH 7.5, 10% sucrose, 50 mM NaCl, 5 mM DTT, 0.5 mM PMSF and 5 mM benzamidine hydrochloride). All of the following steps were done at 4° C.

The cells were lysed by 1 pass through a French pressure cell at 15000 psi and cell debris removed by low speed centrifugation at 20000×g in a Beckman JALO or JA20 rotor. The resulting supernatant, which contained the GT'ase protein, was fractionated with 0.08% (w/v) polyethyleneimine cellulose in order to precipitate and remove nucleic acids and associated binding proteins. The supernatant was further fractionated by ammonium sulfate precipitation. Protein precipitating between 35 and 55% ammonium sulfate saturation (between 0.193 g per mL and 0.326 g/mL of solution) was redissolved and dialysed vs. a buffer containing 25 mM Hepes-KOH pH 7.5, 10 mM NaCl, 0.5 mM EDTA, 0.5 mM DTT, 50 glycerol, 1 mM benzamidine hydrochloride and 0.2 mM PMSF (Buffer C). This dialyzed fraction containing the GT'ase was applied to a Q-Sepharose anion exchange column which had been equilibrated with Buffer C. After a 4 column volume buffer C wash, bound protein was eluted using a linear gradient of 10 to 500 mM NaCl in Buffer C. Fractions containing the GT'ase were identified, precipitated with ammonium sulfate as above, redissolved and dialysed in a buffer containing 25 mM HEPES pH 7.5, 75 mM NaCl, 5? glycerol, 0.5 mM DTT (Buffer D).

This Q-Sepharose fraction was applied to a Heparin-Sepharose affinity column equilibrated in buffer D. The column was washed with 3 column volumes of Buffer D, and bound protein eluted using a linear gradient of 75 to 500 mM NaCl in buffer D. Major contaminants did not bind to the Heparin-sepharose column and eluted in the flow-through fractions, whilst GT'ase was retained. Fractions containing GT'ase were identified, pooled, aliquoted and stored frozen at −80° C. until needed.

The reaction mechanism of GT'ases permits two types of activity assays to be performed. Both assays have been adapted from examples found in the literature (Shibagaki et al., 1992, supra; Yamada-Okabe et al., 1996, supra; Itoh et al., 1984, supra). The first assay detects the formation of an enzyme-GMP covalent intermediate (reaction 2a) and is routinely used to monitor GT'ase purification.

Protein samples containing the GT'ase (i.e. lysates, column fractions) are incubated (5 min. at 37° C.) with 10 μCi of α³²P-GTP (0.3 μM in a 10 μL reaction) in reaction buffer (25 mM HEPES-KOH, 10% glycerol, 50 mM KOAc, 3 mM Mg(OAc)₂, 5 mM dithiothreitol) to form an enzyme-GMP covalent intermediate as per reaction 2a in FIG. 2. Electrophoresis buffer containing SDS is added to quench the reaction and the sample is boiled and analyzed by SDS-PAGE and autoradiography.

The second assay type detects the formation of a 5′-5′ guanylated RNA (reaction 2b in FIG. 2) from an appropriate acceptor RNA containing a diphosphate 5′ end. This assay incorporates both steps of the GT'ase mechanism shown in reaction 2, (FIG. 2). It requires an RNA substrate with a diphosphate 5′ end, which was produced using the 5′ RNA TP'ase activity of the vaccinia virus capping enzyme (Gibco BRL). Treatment of 5′ triphosphate RNA (produced in vitro using T7 RNA polymerase and an appropriate plasmid) yielded the 5′ diphosphate RNA substrate. This RNA was incubated (30 min. 37° C.) with the GT'ase and α³²P-GTP in reaction buffer to synthesize unmethylated, capped RNA (GpppGpN(pN)ₙ). Detection (and quantitation) of the reaction product was done by thin layer chromatography, essentially as described for the MT'ase assay below.

### 8. EXAMPLE

Purification and Assay of Saccharcomyces cerevisiae Methyltransferase

As the MT'ase was expressed as an N-terminal hexa-histidine fusion protein, immobilized metal affinity chromatography (IMAC) (Porath, 1992, Prot. Express. and Purif. 3:263-281) provided the major purification step. Essentially the same procedure was also used to purify N-terminal Hexa-histidine fusions of the C. albicans MT'ase, the human capping enzyme, and the human MT'ase that were expressed in E. coli. Purification was monitored by SDS-PAGE and activity assay. The overexpressed protein was soluble and active throughout the purification procedure.

The strains expressing these proteins were grown in LB medium at 37° C. and induced to express the cloned protein by addition of IPTG to 1 mM. After harvest, the cells were resuspended to 20% (w/v) in a buffer containing 50 mM Tris-Cl pH 7.5, 5% glycerol, 100 mM NaCl, 0.5 mM β-mercaptoethanol, 0.05% triton X-100, 1 mM PMSF and 5 mM benzamidine hydrochloride. All of the following steps were done at 4° C. Cells were lysed by 1 pass through a French pressure cell at 15000 psi. The bulk of the cell debris was removed by low speed centrifugation at 20000×g in a Beckman JA10 or JA20 rotor. Membrane vesicles and ribosomes were removed by ultracentrifugation at 45000 rpm for 1.5 hrs. The resulting supernatant, which contains the desired overexpressed protein, was applied to a TALON (Clontech) immobilized metal affinity column (IMAC) of the appropriate size. The column was then washed with a buffer containing 50 mM Tris-Cl pH 7.5, 5% glycerol, 100 mM NaCl, 0.5 mM β-mercaptoethanol, 25 mM Imidazole and 1 mM Mg(OAc)₂. This low stringency wash removed loosely bound protein contaminants. The bound protein was eluted in a buffer containing 50 mM Tris-Cl pH 7.5, 5% glycerol, 100 mM NaCl, 0.5 mM β-mercaptoethanol, 150 mM Imidazole and 1 mM Mg(OAc)₂. Eluted protein solution was dialysed vs buffer containing 50 mM Tris-Cl pH 7.5, 5% glycerol, 300 mM NaCl, 1 mM Mg(OAc)₂ and 1 mM DTT in order to remove imidazole before storage at −80° C. in small aliquots.

Assay of the ABD1 MT'ase measures the addition of a methyl group, derived from S-adenosyl methionine, to a capped-unmethylated RNA substrate (reaction 3 in FIG. 2) and was adapted from assays described in the literature (Wang et al., 1997, supra). The RNA substrate was produced using the 5′ RNA TP'ase and GT'ase activities of the vaccinia virus capping enzyme. RNA produced in vitro using T7 RNA polymerase was incubated with vaccinia virus capping enzyme and α³²P-GTP in reaction buffer in order to produce a radiolabeled capped-unmethylated RNA substrate (G³²pppGpN(pN) ). The RNA was purified in order to remove free α³²P-GTP and the vaccinia capping enzyme. The labeled RNA substrate (1 pmol, 5000 cpm) was incubated (5 min, 37° C.) with the S. cerevisiae MT'ase in reaction buffer +50 μM S-adenosyl methionine in order to synthesize methylated, capped RNA. After 5 minutes at 37° C., the reaction mixture was acidified to pH 5.5 with sodium acetate and digested with P1 nuclease in order to yield nucleosides and the cap dinucleotides GpppG and Me-7-GpppG. Then, the reaction mixture was applied to polyethyleneimine thin layer chromatography plates and developed in 0.3 M ammonium sulfate. After development the TLC plates were exposed to X-ray film for analysis. Autoradiogram of a TLC assay of 10-fold dilutions of the purified ABD1 MT'ase showed that the purified MT'ase protein can convert >90% of the capped unmethylated RNA to the methylated form. As described above, this TLC assay system can also be used for analysis of GT'ase assays.

### 9. EXAMPLE

Reconstitution of a Complete Capping Assay Using Purified Holoenzyme

The feasibility of designing a complete cap synthesis assay using all three of the required enzymes and an unmodified RNA substrate was demonstrated in an experiment using capping holoenzyme (2-subunit TP'ase/GT'ase complex) partially purified from C. albicans and the S. cerevisiae MT'ase. These enzymes synthesized a fully capped mRNA that was subsequently used to direct the synthesis of a functional reporter protein (firefly luciferase) in an in vitro translation reaction.

Small quantities of the C. albicans capping holoenzyme were partially purified as described for the S. cerevisiae enzyme (Itoh et al., 1984, J. Biol. Chem. 259:13923-13929). Purification was monitored by assay for GT'ase-GMP covalent intermediate formation and was carried through for the first 5 Fractions (lysate, ammonium sulfate precipitation, polyethyleneimine precipitation, Q Sepharose anion exchange and CM Sepharose cation exchange chromatography). In order to determine whether the TP'ase activity was co-purifying with the observed GT'ase activity, a linked capping/in vitro translation assay was constructed. Varying quantities of the C. albicans capping holoenzyme and a saturating amount (0.4 pg) of the purified MT'ase were incubated (15 min, 37° C.) with 2 μg of an RNA encoding a firefly luciferase reporter gene containing a triphosphate 5′ end (i.e. 5′ pppGpNp(FLuC)pN) in translation lysate buffer (Iizuka et al., 1994, Mol. Cell. Biol. 14:7322-7330) +50 μM S-adenosyl methionine.

The action of all three enzymes is required to convert the RNA substrate into a translatable mRNA (5′ Me-7-GpppGpNp(FLUC)pN). This reaction mixture was then added to in vitro translation reactions using S. cerevisiae translation lysates (Iizuka et al., 1994, supra) and further incubated, in order to translate the capped mRNA synthesized into functional luciferase protein. Capping activity (as measured by activity of luciferase reporter in relative light units (RLU)) was stimulated ˜25-fold with the addition of increasing amounts of C. albicans capping holoenzyme, indicating that 5′ mRNA caps were being synthesized and that the TP'ase was present. The concentration of the S. cerevisiae MT'ase was kept constant throughout the experiment (0.4 μg per assay).

The S. cerevisiae in vitro translation system used in this experiment has been shown to be dependent on the presence of the 5′cap structure. As confirmation, a series of controls were performed to verify cap dependence. Accordingly, uncapped and unmethylated RNA substrates (5′ pppGpNp(F_{Luc})pN and 5′ GpppGpNp(FLUG)pN) were translated poorly by in vitro translation lysates (<5% maximal signal) in the absence of capping enzymes.

The results demonstrated the feasibility of designing a full cap synthesis assay using all three of the required enzymes and an unmodified RNA substrate. Thus, given sufficient quantities of protein (overexpressed in E. coli) and a reliable detection system, capping assays are amenable to high-throughput screening formats. Given the disclosure herein, any of the three capping enzymes from either S. cerevisiae or C. albicans or human (or other organisms), in any combination, can be cloned, expressed and purified for use in the capping assays of the invention.

### 10. EXAMPLE

Cloning and Sequencing of a C. albicans mRNA Triphosphatase

A short partial sequence (approximately 400 nucleotides) of the C. albicans triphosphatase gene (CET1) was available on the world wide web by accessing the following sites: http://alces.med.umn.edu/Candida.html (click on “genes,” which takes you to) http://alces.med.umn.edu/bin/genelist?genes (all the cloned Candida sequences, click on genes of interest). A fragment corresponding to this partial sequence was obtained by PCR and used to clone the complete C. albicans CET1 gene as described in more detail below.

The following PCR primers were made and used to amplify from C. albicans genomic DNA a fragment corresponding to the published 400 nucleotide fragment using standard polymerase chain amplification techniques:

Primer 1a GGGCATGCAAGTGGAAG (SEQ ID NO:5); and

Primer 2a GGGTACCCAATGACCCTAG (SEQ ID NO:6).

The resulting amplified fragment was then inserted into the standard cloning vehicle pBluescript. Restriction enzyme digests and sequencing confirmed that the isolated fragment was truly the same as the published sequence.

From blast searches, it was believed that the isolated Candida CET fragment was homologous to the 3 prime end of the S. cerevisiae gene. From homology studies with the S. cerevisiae gene, we hypothesized that a Psh AI restriction enzyme site at the 3′ end of the sequence of the isolated Candida DNA fragment would be ^{˜}1.5 kb downstream of the beginning of the CET1 gene. Accordingly, the cloned PCR fragment was used to probe Southern blots of Candida genomic DNA digested with Psh AI and different restriction enzymes in an attempt to identify an enzyme that generated a >1.5 kb fragment that should contain 5′ coding sequence. Sph I digestion resulted yielded a Candida genomic fragment ^{˜}1.6 kb upstream of the Psh AI site.

Additional Southern blots were performed using Sph I and a number of different enzymes. An approximately 2.8 kb Sph I-Xba I Candida genomic fragment was identified that was thought to encompass the complete Candida CET1 gene.

Candida albicans genomic DNA (10 μg) was digested overnight with Sph I and Xba I, and the liberated DNA fragments were separated by electrophoresis through a low melting point agarose gel. A piece of the agarose gel was isolated that corresponded to the ^{˜}2.8 kb region (identified by known DNA molecular weight markers). DNA was purified from the agarose gel fragment following standard methods.

The Southern blots had demonstrated previously that there were no sites for the restriction enzymes Bgl I, Eag I, Sal I, Spe I and Xho I in the CET1 encompassing Sph I-Xba I fragment. So, these enzymes were used to reduce the number of additional ^{˜}2.8 kb Sph I-Xba I fragments from the Candida genomic digest that didn't encode CET1.

After this second digest the DNA was ligated into Sph I-Xba I digested pUC118 and transformed into E. coli DH5α. From the ^{˜}75 transformants, one appeared to contain the expected Candida CET1 encompassing Sph I-Xba I fragment (determined by restriction digest analysis). This ^{˜}2.8 kb fragment was sequenced and shown to contain a 3.lkb Sph I-Xba I fragment that from similarity searches appeared to indeed encode a gene homologus to S. cerevisiae CET1.

The Candida CET1 gene open reading frame is 1563 bp in length and encodes 521 amino acids. At the amino acid level, there is ^{˜}27% identity and ^{˜}60% homology between the Candida and Saccharomyces CET1 coding sequences. The complete nucleotide sequence of the Sph I-Xba I fragment from the genome of C. albicans that contains the mRNA triphosphatase gene, CET1, is provided in SEQ ID NO:1. Translation is expected to begin at the AUG codon at nucleotide residue 354 and continue to the stop codon at nucleotide residue 1914. The deduced amino acid sequence encoded by the CET1 gene is displayed in SEQ ID NO:2.

### 11. EXAMPLE

Cloning and Sequencing of a C. albicans mRNA methytransferase

A similar strategy was used to clone the C. albicans ABD1 gene. Like the CET1 gene, a partial sequence of about 300 nucleotides thought to correspond to a portion of the ABD1 gene was publicly available on the world wide web at the same sites noted above. PCR primers made for amplifying an approximately 300 base pair fragment containing this published sequence were as follows:

Primer 1b GGGCATGCAATGTTCCTGAGTAT (SEQ ID NO:7); and

Primer 2b GGGTACCAATGCNACNGCTTC (SEQ ID NO:8).

After amplification of the desired fragment from Candida genomic DNA, the fragment was inserted into the standard cloning vehicle pUC118. Restriction enzyme digests and sequencing confirmed that the isolated fragment was the same as the published sequence.

From blast searches it was believed that the isolated Candida ABD1 fragment was homologous to the 3 prime end of the S. cerevisiae gene. In an analogous manner to the CET1 protocol described above, restriction enzyme digests and Southern blots were used to identify an ^{˜}2.4 kb SacI-SpeI fragment which should encompass the complete Candida ABD1 gene.

Candida albicans DNA (10 μg) was digested overnight with SacI and SpeI, and the liberated DNA fragments were separated by electrophoresis through a low melting point agarose gel. A piece of the agarose gel was isolated that corresponded to the ^{˜}2.4 kb region (identified by known DNA molecular weight markers). DNA was purified from the agarose gel fragment following standard methods.

The Southern blots had demonstrated that the were no sites for the restriction enzymes Xba I, Xho I, Hind III, Kpn I, and Sph I in the ABD1 encompassing SacI-SpeI fragment. So these enzymes were used to reduce the number of additional ^{˜}2.4 kb Sac I-Spe I fragments from the Candida genomic digest that didn't encode ABD1. After this second digest, the DNA was ligated into Sac I-Spe I digested pBLUESCRIPT KSII and transformed into E. coli DH5α. From ^{˜}350 transformants, one appeared to contain the expected Candida ABD1 encompassing Sac I-Spe I fragment (determined by Southern blot analysis). This ^{˜}2.4 kb fragment was sequenced and shown to contain a 2.4 kb Sac I-Spe I fragment that from similarity searches encoded a gene homologous to S. cerevisiae ABD1.

The nucleotide sequence of the Sac I-Spe I fragment from the genome of C. albicans that contains the ABD1 methyltransferase gene is shown in SEQ ID NO:3. SEQ ID NO:4 illustrates the deduced amino acid sequence of the ABD1 gene transcript. The Candida ABD1 gene open reading frame is 1425 bp in length and encodes 475 amino acids. Translation is predicted to initiate with the AUG codon at nucleotide position 236, and to terminate at the TAG codon at position 1661. There is ^{˜}40% identity and ^{˜}67% homology between the Candida and Saccharomyces ABD1 coding sequences.

### 12. EXAMPLE

Purification of S. cerevisiae and C. albicans Triphosphatase

The E. coli strain expressing the cloned S. cerevisiae CET1 (TP'ase) protein was grown in LB medium at 37° C. and induced to express the cloned protein by the addition of IPTG to 1 mM. After harvest, the cells were resuspended to 20% (w/v) in a buffer (Buffer A) containing 50 mM Tris-Cl pH 7.5, 10% glycerol, 50 mM NaCl, 0.5 mM DTT, 1 mM PMSF and 5 mM bezamidine hydrochloride. All of the following steps were done at 4° C. The cells were lysed by 1 pass through a French pressure cell at 15000 psi. Bulk cell debris was removed by low speed centrifugation at 20000×g in a Beckman JA10 or JA20 rotor. The resulting supernatant, which contains the TP'ase protein, was fractionated with 0.1% (w/v) polyethyleneimine cellulose in order to precipitate and remove nucleic acids and associated binding proteins. The supernatant was further fractionated by ammonium sulphate precipitation. Solid ammonium sulfate was added to 43% of saturation (0.243 g per mL of solution) and the precipitate, containing the TP'ase protein, was redissolved and dialysed against buffer A. This dialyzed fraction was applied to a Q-Sepharose anion exchange column which had been equilibrated with buffer A. After a 4 column volume buffer A wash, the protein was eluted using a linear gradient of 50 to 500 mM NaCl in buffer A. Fractions containing the TP'ase were identified, precipitated with ammonium sulfate, redissolved and dialysed in buffer A. This Q-sepharose fraction was applied to a CM-sepharose cation exchange column equilibrated in buffer A, and the column washed with 3 column volumes of buffer A. The TP'ase protein does not bind to the CM-sepharose column and eluted in the flow-through fractions, while major contaminant were retained on the column. The fractions containing the TP'ase were identified, pooled, aliquoted and stored frozen at −80° C. until needed.

The C. albicans CET1 protein is purified using an approach similar to that described above. Comparison of the predicted pI values for the two proteins (S. cerevisiae CET1 pI=5.26; C. albicans CET1 pI=7.93) suggests that the 2 proteins exhibit different behavior during ammonium sulfate precipitation and on the ion exchange resins. However, the overall approach of ammonium sulfate fractionation followed by anion exchange followed by cation exchange is the same for both proteins.

### 13. EXAMPLE

Reconstitution of a Complete Fungal Capping Assay Using Recombinantly Expressed Proteins

A complete fungal capping reaction was reconstituted, for the first time, using recombinantly expressed proteins for all three capping enzymes. S. cerevisiae CET1 (TP'ase) protein, S. cerevisiae ABD1 (MT'ase) protein, and C. albicans CGT1 (GT'ase) protein were recombinantly expressed and purified as described above (in Sections 7, 8 and 12). The RNA substrate (50 picomoles) was an RNA encoding a 139-base fragment of a Renilla luciferase reporter gene containing a triphosphate end, as described above in Section 9. Purified S. cerevisiae CET1 (TP'ase) protein, purified S. cerevisiae (MT'ase) protein, and purified C. albicans CGT1 (GT'ase) protein (10 ng of each) were incubated with the RNA substrate for 15 min at 37° C. in buffer (50 mM Tris-HCl pH 7.5; 15 mM NaCl; 1 mM DTT; 1.5 mM GTP; 4 mM MgCl₂; 0.05 mg/ml BSA) +50 μM ³H—S-adenosyl methionine (1 μCi, 1000 cpm/pmol).

The action of all three enzymes is required to convert the RNA substrate into a translatable (and, in this case, tritiated) mRNA (5′ ³H—Me-7-GpppGpNpNₙ). Incorporation of ³H into the substrate RNA was measured using the GFC assay described below. Capping activity (as measured by incorporation of tritiated label) was dependent upon addition of purified capping enzymes.

### 14. EXAMPLE

Assays for mRNA Triphosphatase

Capping triphosphatase enzymes catalyze the hydrolysis of the γ-phosphate of pppRNA to liberate free inorganic phosphate (see FIG. 2, step (1)). The assays below monitor the release of inorganic phosphate from a 5′ triphosphate end labeled substrate RNA molecule.

14.0.1 Components

The substrate RNA is produced using either E. coli RNA polymerase, a random DNA template and γ-³²P-ATP, or T7 RNA polymerase, γ-³²P-GTP and a suitable linearized plasmid DNA template containing a phage T7 promoter.

In the former case, the RNA substrate is a 5′ triphosphate end labeled poly(A), typically 200 to 2000 bases in length. In the latter case, the RNA substrate is a 5′ triphosphate end labeled RNA with a specific sequence as directed by the plasmid. In either case the product RNA is purified by repeated EtOH precipitation or G-25 gel filtration chromatography (spin column) in order to remove unincorporated nucleotides. For non-radioactive detection methods, the substrate RNA is not labeled.

In this example, template was produced using a SmaI linearized pRG166 vector. pRG166 directs the production of a luciferase-encoding mRNA using the T7 transcription mMachine system (Ambion). For optimal translation in a yeast in vitro translation system, DNA encoding the original luciferase 5′UTR (untranslated region) was replaced with DNA encoding the 5′UTR from the highly expressed yeast gene ADH1. DNA encoding this modified luciferase construct (under the control of DNA encoding a T7 transcription promoter) was placed in the standard cloning vector pUC118 to generate vector pRG166. However, any appropriate vector can be used.

TP'ase for use in the assays can be fungal, viral or human derived TP'ase's.

14.0.2 Assay Conditions

The human capping enzyme triphosphatase (typically 100 ng) was assayed using 50 to 200 pmol of substrate 5′ ³²pppNpN . . . RNA in a buffer containing 25 mM Tris-Cl, pH 7.5, 0.5 mM DTT. Reactions were carried out in a volume of 10 to 20 μL for 10 min at 37° C. Reactions were stopped by the addition of MgCl₂ to 10 mM and 40 μg of carrier poly(A) RNA. Total RNA was precipitated by the addition of 0.5 mL of 5% TCA and collected on Whatman GF/C glass fiber filters pre-wetted with 0.2 M sodium pyrophosphate, 1 M HCl. The filters were washed twice with 3 mL of 0.2 M sodium pyrophosphate, 1 M HCl and once with 3 mL of 95% ethanol. Dried filters were then counted in the scintillation counter.

Fungal TP'ase (both the Candida and Saccharomyces enzymes) assay was done in the same manner as for the human TP'ase except that the reaction buffer contains 25 mM Tris-Cl, pH 7.5, 50 mM KOAc, 4 mM Mg(OAc)₂ and 0.5 mM DTT. Reactions were carried out in a volume of 10 to 20 μL for 5 min at 37° C. For detection via a glass-fiber filter binding assay (GFC detection), the reaction was stopped by addition of 1 drop (from a pasteur pipette) of 0.2 M sodium phosphate, 0.2 M EDTA. Carrier DNA (5 μg) and 0.5 mL of ice cold 10% TCA were added to precipitate the nucleic acids.

For detection by TLC, the reactions were quenched by the addition of 1 μL of 0.5 M EDTA. The reaction volume was kept to a minimum since the TLC analysis phase is not amenable to large volumes. When the liberated inorganic phosphate was analyzed by non-radioactive methods, the reactions were quenched by heat inactivation at 95° C. for 3 minutes.

14.0.3 Detection

For the GFC binding assay, after incubation on ice for 5 to 10 minutes, the reaction tubes were filled with 0.1 M sodium pyrophosphate, 1 M HCl and vacuum filtered through Whatman GF/C glass-fiber filters which had been pre-soaked in the same solution. Filters were rinsed twice with 3 mL of the NaPPi/HCl solution in order to wash away radioactive phosphate that had been cleaved from the RNA substrate, and finally with 3 mL of 95% EtOH in order to facilitate drying. Filters were dried under a heat lamp for 5 min., then the retained, radiolabeled RNA was quantitated by scintillation counting.

For reactions analyzed by polyethyleneimine cellulose thin layer chromatography (TLC), portions of each reaction containing 3000 cpm were spotted onto the TLC plates (3 mL per application with drying in between applications using a hair dryer). The TLCs were developed in 0.5 M sodium phosphate pH 3.4, wrapped in saran wrap, and exposed to X-ray film overnight. Resulting autoradiograms provided a qualitative assay result. Semi-quantitative results were obtained by scanning the autorad using a calibrated flatbed scanner and the appropriate image analysis software (e.g., NIH IMAGE vl.61). More precise quantitation was achieved using a phosphorimager, or by scintillation counting of the radioactive spots after they were cut out of the TLC plate using the autorad as a guide. Unhydrolysed substrate RNA remained at the origin during the TLC analysis while free phosphate migrated to near the top of the chromatogram. The TLC assay is described in the literature (see Yue et al., 1997, supra; Tsukamoto et al., 1997, supra; Takagi et al., 1997, Cell 89:867-873; Ho et al., 1998, J. Biol. Chem. 273:9577-9585; and Shuman et al., 1980, J. Biol. Chem. 255:11588-11598).

When non-radioactive RNA substrate was used, the free phosphate produced in the reaction was assayed, for example, using the EnzCheck™ Phosphate assay kit (Molecular probes, Eugene, Oreg., Cat # E-6646). This assay detects between 2 and 150 μM Pi per assay and was monitored spectrophotometrically at 360 nm using the Molecular Dynamics SPECTRAmax plate reader. The basis of the assay is the conversion of 2-amino-6-mercapto-7-methylpurine riboside and Pi to 2-amino-6-mercapto-7-methylpurine and ribose-1-phosphate by the enzyme Purine nucleotide phosphorylase. The reaction product, 2-amino-6-mercapto-7-methylpurine, absorbs maximally at 360 nm, while the substrate absorbs maximally at 330 nm.

### 15. EXAMPLE

Assays for mRNA Guanylyltransferase

15.1 Enzyme-GMP Gel Assay

This assay targets the first part of the overall guanylyltransferase reaction, the formation of an enzyme-GMP covalent intermediate, and is a modification of that described in the literature (see Yue et al., 1997, Proc. Natl. Acad. Sci. USA, 94:12898-12903; Shibagaki et al., 1992, J. Biol. Chem., 267:9521-9528; Yamada-Okabe et al., 1996, supra; Ho et al., 1998, J. Biol. Chem., 273:9577-9585; Itoh et al., 1984, J. Biol. Chem., 259:13923-13929). Samples containing the guanylyltransferase were incubated with 0.25 ,μCi of α-³²P-GTP in an appropriate buffer as described above in Section 7 for 5 minutes at 37° C. in a 10 μL reaction. The reaction was quenched by the addition of 5 μL of 3× SDS electrophoresis sample buffer and boiled for 5 minutes. Subsequent SDS-PAGE and autoradiography (from 10 min. to 1 hour) provided a qualitative assay for the guanylyltransferase protein during chromatography.

15.2 a-³²P-GMP Transfer to Diphosphate 51-ended RNA

This assays includes both parts of the guanylyltransferase reaction (see steps 2(a) and (b) in FIG. 2). The RNA substrate was not radiolabeled.

15.2.1 α-³²P-GMP Transfer to Diphosphate 5′-ended RNA

In a first type of assay, the RNA substrate was produced with a triphosphatase to ensure the presence of a diphosphate 5′ end using a scaled-up version of the assay described in Section 15.1 above, followed by purification of the RNA by either repeated EtOH precipitation or G-25 gel filtration chromatography (spin column).

For the assay, samples containing the guanylyltransferase were incubated with 0.25 μCi of α-³²P-GTP and the substrate RNA in an appropriate buffer for 5 minutes at 37° C. in a 10 μL reaction. The reaction was quenched by heat inactivation at 95° C. for 3 minutes, and analyzed either by a glass-fiber filter binding assay as described above (quantitative assay) or by a PEI cellulose TLC assay. For the PEI cellulose TLC assay, 1 AL of 0.55 M Na(OAc), pH 5.5 and 1 μL of P1 nuclease (5 mg/mL) were added to the quenched reactions and incubated at 37° C. for 1 hour in order to digest the RNA. The samples were spotted onto a PEI cellulose TLC plate as described above and developed in 0.4 M ammonium sulfate. Autoradiography and quantitation were done as described above.

The order of migration of reaction products from the bottom (origin) of the TLC to the top was: origin (unreacted substrate), GTP, GpppG, Me-7-GpppG and Pi.

15.2.2 α-³²P-GMP Transfer to Diphosphate 51-ended RNA

In lieu of performing the GT'ase and TP'ase reactions separately, the assay can also be performed in one reaction vessel. Unlabeled RNA substrate was incubated in the appropriate buffer (See Section 7 above) along with purified triphosphatase, guanyltransferase and a-labeled GTP. TLC separation and analysis was as described above.

15.3 Linked Pyrophosphatase Assay

This assay can be performed as described in Section 15.2, but using GTP instead of a-³²P-GTP. The guanylyltransferase reaction is also carried out in the same manner as described above, however, the reactions are quenched by heat inactivation at 95° C. for 3 minutes. The pyrophosphate (PPi) produced in the reaction is assayed using the EnzCheck~m Pyrophosphate assay kit (Molecular Probes, Eugene, OR, Cat # E-6645). This assay is essentially the same as the phosphate assay described above (Section 14) but additionally includes a pyrophosphatase which converts the PPi into 2 equivalents of Pi which are then assayed with of 2-amino-6-mercapto-7-methylpurine riboside and Purine nucleotide phosphorylase as described above. By monitoring the reaction spectrophotometrically at 360 nm using the Molecular Dynamics SPECTRAmax plate reader, between 1 and 75 μM PPi is detected per assay.

### 16. EXAMPLE

Assays for mRNA Methyltransferase

Assays to measure mRNA methyltransferase rely upon the detection, and optionally quantitation, of the transfer of a methyl group to guanylylated RNA.

16.1 TLC Assay: ³²P-Based

The TLC assay was a modification of that described (see Mao et al., 1995, Mol. Cell. Biol., 15:4167-4174; Ping-Wang and Shuman, 1997, J. Biol. Chem., 272:14683-14689). This assay used a ³²P-radiolabeled RNA substrate that was produced using either the human capping enzyme (prepared as described above in Section 8), the Vaccinia capping enzyme (commercially available GIBCO/BRL) or the purified yeast triphosphatase and guanylyltransferase. These enzymes mixed with a T7 RNA polymerase transcribed RNA and α-³²P-GTP in an appropriate buffer produced the required RNA. The RNA was then purified.

For the methyltransferase assay, samples containing the methyltransferase were incubated with the substrate RNA (3000 cpm) in an appropriate buffer, containing 50 μM S-Adenosyl methionine, for 10 minutes at 37° C. in a 10 μL reaction. The reaction was quenched by heat inactivation at 95° C. for 3 minutes, and analyzed by the PEI cellulose TLC assay as described above.

16.2 Glass-fiber Filter Binding Assay: ³H-Based

This assay uses an unlabelled RNA produced using the Ambion mMessage mMachine RNA synthesis kit (Ambion, Inc., Cat # 1344), but with substitution of the un-methylated cap analogue (GpppG, Ambion, Inc, Cat #8035) for the methylated cap analogue (Me-7-GpppG) which is normally supplied with the kit. Using an appropriate plasmid containing a phage T7 promoter, this produces an un-methylated capped RNA that is the methyltransferase substrate. This RNA was incubated with the methyltransferase in a suitable buffer which contained S-Adenosyl-L-[methyl-³H]methionine (Amersham Pharmacia Biotech, Cat # TRK236) and unlabelled S-Adenosyl-L-methionine to a final concentration of 50 μM, in a 10 μL reaction. The reaction was incubated for 10 minutes at 37° C., then quenched as described above and analyzed by the glass-fiber filter binding assay (quantitative assay).

16.3 Linked In Vitro Translation Assay

This assay uses an unlabelled Luciferase-encoding RNA substrate produced as described in Section 14 above (from plasmid pRG166/SmaI- although other reporter genes besides luciferase can also be used). The methyltransferase reaction is also carried out in the same manner as in Section 8 (methyltransferase+2 μg RNA+buffer), except that no radiolabel is used, only 50 AM cold S-Adenosyl Methionine. After the reaction is quenched by heat inactivation at 95° C. for 3 minutes, 2.5 μL (0.5 μg RNA) are added to a standard S. cerevisiae in vitro translation assay. Alternatively, a C. albicans in vitro translation assay can be used. Amount of Luciferase signal obtained from the methyltransferase reactions compared to fully methylated and un-methylated controls correlates with the extent of substrate methylation. Luciferase levels are measured by addition of a luciferin reagent (Analytical Bioluminescence, Ann Arbor, Mich.). Light output in relative light units (RLU's) was detected using a luminometer (Dynatech ML3000). Additionally, assay sensitivity is enhanced by using a non-polyA mRNA substrate.

### 17. EXAMPLE

High Throughput Assays for Inhibition of mRNA Capping

Provided by the invention, for the first time, are screens for compounds that affect fungal capping using purified enzymes for all three fungal capping reactions.

17.1 Scintillation Proximity Assay (SPA) 1-Hybridization Assay

This assay targets all 3 fungal capping enzymes and uses Scintillation Proximity Assay technology (commercially available from Amersham, Arlington Heights, Ill.). The 3 capping enzymes were used to modify an unlabeled RNA substrate to make a product with a ³H-Methyl group incorporated in the last reaction. The RNA substrate is a short (^{˜}100-base) unlabeled 5′ triphosphate terminated RNA transcribed from a defined plasmid template containing a phage T7 promoter. RNA is synthesized from the template using commercially available T7-based transcription kits (e.g. Ambion MegaShortScript). Since the last reaction (MT'ase) is dependent on the previous 2 reactions, signal is dependent on the activity of all 3 enzymes. Presence or absence of an RNA cap structure is detected by hybridization of the RNA product to a synthetic, complimentary 3′ biotinylated capture oligonucleotide bound to streptavidin conjugated SPA beads, thus bringing the ³H radiolabel close enough to the SPA bead to produce a measurable light signal. Streptavidin beads are routinely used in SPA assays. Compounds that cause a signal loss in the assay are scored as hits.

17.2 Direct Scintillation Proximity Assay

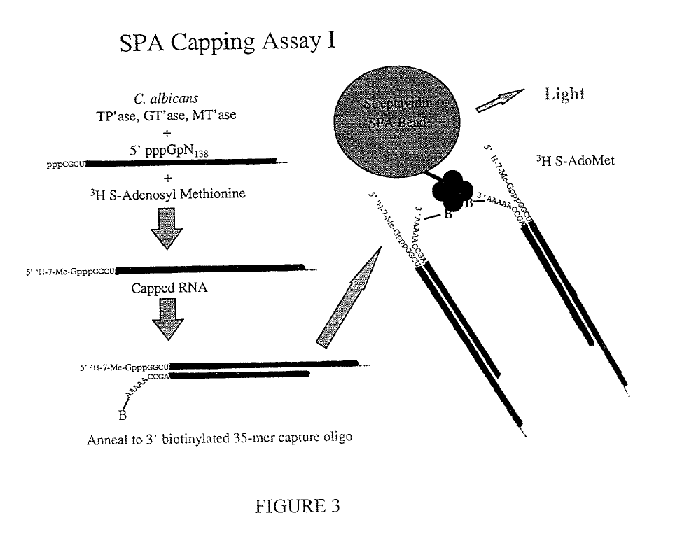

In an alternative to the above-described SPA based assay, a similar RNA template is synthesized using commercially available T7-based transcription kits (e.g. Ambion MegaShortScript) but modified to include biotinylated ribonucleotides (Biotin-16-UTP or Biotin-14-CTP) in the transcription reaction. Thus, biotin is incorporated into the RNA chain and can be used to capture the RNA template on Steptavidin SPA beads. FIG. 3 presents a diagram of the assay. Since the last reaction (MT'ase) is dependent on the previous 2 reactions, the signal is again dependent on the activity of all 3 enzymes. Presence or absence of a radioactive RNA cap structure is detected by direct hybridization of the capped biotinylated RNA product to streptavidin conjugated SPA beads, thus bringing the ³H radiolabel close enough to the SPA bead to produce a measurable light signal. Streptavidin beads are routinely used in SPA assays. Compounds that cause a signal loss in the assay are scored as hits.

17.3 Filter-Binding Assay

The capping assay part of this screen is identical to the SPA assays above. Detection of the assay products was achieved by direct scintillation counting. Reactions were quenched with TCA in order to precipitate the RNA, then filtered through glass fiber filters in a 96-well format. All of the RNA in the assay bound to the filter, while the unincorporated radiolabel flowed through the filter during subsequent wash steps. As in the previously described assay, compounds that cause a signal loss are scored as hits. This assay has been performed using purified TP'ase and MT'ase from S. cerevisiae, and purified GT'ase from C. albicans.

17.4 Linked In Vitro Translation Assay

This non-radioactive assay uses a fungal in vitro translation (IVT) assay as a detection system for cap synthesis. IVT is dependent on the presence of a fully capped RNA for maximal translation activity. The RNA substrate is a T7 RNA polymerase transcript encoding a reporter gene such as luciferase (firefly or Renilla) or green fluorescent protein (see U.S. Pat. Nos. 5,491,084, 5,804,387, 5,777,079 and 5,741,668). Use of a non-polyA RNA substrate increases the cap-dependent change in signal. Again, as in the above assays, compounds that cause a signal loss are scored as hits.

17.5 Scintillation Proximity Assay (SPA)- eIF4E-Linked SPA Assay

This assay targets all 3 fungal capping enzymes and also uses the Scintillation Proximity Assay technology (Amersham). The 3 capping enzymes act on an internally radiolabeled RNA substrate to make a product RNA with a complete 5′ cap structure. RNA substrate is synthesized using T7 RNA polymerase, an appropriate a-labelled ribonucleotide triphosphate and a DNA template derived from a plasmid containing a phage T7 promoter. Detection of the fully capped RNA is accomplished using eIF4E, the eucaryotic cap binding protein which specifically recognizes the Me-7-GpppN . . . mRNA cap structure (cap-specific DNA aptamers, antibodies or peptides can also be used). Positive signal depends upon the functioning of all 3 enzymes in the capping assay since eIF4E binds uncapped or partially capped RNA's with significantly lower affinity than the fully capped form.

The detection phase of the assay takes a variety of final forms depending on the type of SPA bead used and the type of modified eIF4E used. For example, biotinylated eIF4E (produced chemically or via in vitro biotinylation of a short biotinylation consensus peptide fused to eIF4E) can be used to capture capped RNAss and bring them into proximity to streptavidin SPA beads. A variation of this assay uses eIF4E produced as a GST fusion and linked to glutathione SPA beads. Streptavidin and glutathione SPA beads are routinely used in SPA assays. Compounds that affected any of the 3 capping enzymes cause a signal loss in the assay and are scored as hits.

17.6 Scintillation Proximity Assay (SPA) 4- Antibody-eIF4E-linked SPA Assay

This assay format is similar to the previous assays (Section 17.4). The only difference is in the detection phase; the actual cap synthesis phase of the overall assay is identical to that described above. The detection phase of the assay uses unmodified eIF4E and an antibody raised against the eIF4E protein (for example Mouse anti eIF4E IgG (Ma4E IgG)). The complex of the capped RNA, eIF4E and the Ma4E IgG is captured using a rabbit anti-mouse IgG SPA bead. Alternatively, Protein A SPA beads are used to capture the RNA-eIF4E-IgG complex. Protein A-, anti-rabbit, anti-mouse and anti-sheep SPA beads are all commercially available from Amersham. Compounds that affected any of the 3 capping enzymes cause a signal loss in the assay and are scored as hits.

17.7 Scintillation Proximity Assay (SPA) 5-RNA Aptamer Linked SPA Assay

This assay is similar to the SPA assay described above in Section 17.1 in that it uses an RNA molecule to bind the capped RNA synthesized in the capping assay. However, the RNA does not act to capture the capped RNA by simple hybridization, but rather via a specific interaction with the Me-7-GpppN . . . cap structure. The sequence of a cap binding RNA aptamer has been produced and was shown to bind to the cap structure with an affinity similar to that of eIF4E (Hailer A. A. and Sarnow P., 1997, PNAS, USA 94:8521-8526). This RNA aptamer is biotinylated and attached to Streptavidin SPA beads as the detection system for capped RNA's produced in the capping assay. Compounds that affect any of the 3 capping enzymes cause a signal loss in the assay and are scored as hits.

17.8 Triphosphatase Assay (SPA or Glass Fiber Filter Binding)

Since the fungal triphosphatase is the most differential (i.e. non-conserved relative to the metazoan triphosphatase) of the 3 capping enzyme targets, an individual screen for this enzyme alone is presented. Although only a SPA example is shown, this assay is also amenable to a glass fiber filter binding assay.

RNA substrate was synthesized using γ-³³P-ATP (or γ-³²P-ATP), E. coli RNA polymerase and a non-specific DNA template. This substrate RNA contained a radiolabel at the 5′ phosphate position. Compounds which adversely affect Triphosphatase activity cause a retention of the radioactive signal and are scored as hits.

17.9 Guanyltransferase and Triphosphatase Double Assay (SPA or Glass Fiber Filter Binding)

The GT'ase and TP'ase reactions can also be screened simultaneously. Biotinylated RNA substrate is produced as described above in Section 17.2. This RNA substrate is then incubated in the appropriate buffer along with purified triphosphatase, guanyltransferase and α-labeled GTP and subjected to different test compounds. Loss of signal in presence of the test compound is scored as a hit.

17.10 Dioxygenin-labeled RNA

Instead of using radiolabeled RNA, the screens outlined above are reconfigured to use digoxigenin labeled RNA. Detection is performed using an ELISA system. Biotinylated eIF-4E or cap binding RNA aptamer are bound to streptavidin coated microtiter plates. Any capped RNA that becomes bound to the eIF-4E or RNA aptamer is detected using a standard immunoassay system such as horseradish peroxidase conjugated anti-digoxigenin antibodies.

### 18. EXAMPLE

Tertiary Screen

Knockout strains of S. cerevisiae which contain deletions of the individual capping enzymes and substitution with the capping enzyme from a different eukaryote have been generated using standard yeast molecular biology techniques. The following strains have been produced:

S. cerevisiae CET1 knockout strain complemented by the S. cerevisiae CET1 gene

S. cerevisiae CET1 knockout strain complemented by the C. albicans CET1 gene

S. cerevisiae CET1 knockout strain complemented by the human capping gene

S. cerevisiae CET1 and CGT1 double-knockout strain complemented by the C. albicans CET1 and CGT1 genes

S. cerevisiae CET1 knockout strain complemented by the C. albicans CET1 and CGT1 genes

S. cerevisiae CGT1 knockout strain complemented by the S. cerevisiae CGTI gene

S. cerevisiae CGT1 knockout strain complemented by the C. albicans CGT1 gene

S. cerevisiae CGT1 knockout strain complemented by the human capping gene

Thus, each group of three strains are identical except for the origin of their CET1 and/or CGT1 gene.

These experiments demonstrate that the C. albicans genes for CET1 and CGT1 can complement the corresponding S. cerevisiae knockout mutants. However, the S. cerevisiae CET1/CGT1 double knockout strain complemented by both the CET1 gene and CGT1 gene from C. albicans grew much better than the S. cerevisiae CET1 single knockout strain complemented by the C. albicans CET1 gene. These results indicate that the C. albicans CET1 gene product interacts more efficiently with the C. albicans CGT1 gene product than with the S. cerevisiae gene product.

Strains are grown in the presence of test compounds. Any compounds that differentially inhibit growth of the strains expressing fungal capping enzymes compared to the strain expressing the human capping enzyme is assumed to be selectively inhibiting the activity of the fungal TPases.

The present invention is not to be limited in scope by the specific embodiments described herein. Indeed, various modifications of the invention in addition to those described herein will become apparent to those skilled in the art from the foregoing description and accompanying figures and sequences. Such modifications are intended to fall within the scope of the appended claims.

Various references are cited herein, the disclosures of which are incorporated by reference in their entireties.

What is claimed is:

1. An isolated polynucleotide, comprising a nucleotide sequence that encodes a polypeptide comprising the amino acid sequence as shown in SEQ ID NO:2.

2. The polynucleotide of claim 1 in which the nucleotide sequence is shown in SEQ ID NO:1.

3. An isolated polynucleotide, comprising a nucleotide sequence that encodes a polypeptide comprising an amino acid sequence selected from the group consisting of residues 173 to 520, residues 206 to 214, residues 283 to 297, residues 438 to 451, and residues 464 to 476 of SEQ ID NO:2.

4. The polynucleotide of claim 3 in which the nucleotide sequence is selected from the group consisting of residues 870 to 1913, residues 969 to 995, residues 1200 to 1244, residues 1665 to 1706, and residues 1743 to 1781 of SEQ ID NO:1.

5. An isolated polynucleotide, comprising a nucleotide sequence of at least 15 nucleotides that hybridizes under stringent conditions to a second polynucleotide having a nucleotide sequence as shown in SEQ ID NO:1 or to the complementary sequence of the second polynucleotide.

6. An isolated polynucleotide, comprising a nucleotide sequence that hybridizes under stringent conditions to a second polynucleotide having a nucleotide sequence as shown in SEQ ID NO:1 or the complementary sequence of the second polynucleotide, and which isolated polynucleotide encodes a naturally-occurring polypeptide.

7. The polynucleotide of claim 1, 3, or 5 which is DNA.

8. The polynucleotide of claim 1, 3, or 5 which is RNA.

9. A recombinant vector containing the polynucleotide of claim 1, 2, 3, 4, 5 or 6.

10. A recombinant expression vector containing the polynucleotide of claim 1, 3, or 5 in which the nucleotide sequence of the polynucleotide is operatively associated with a regulatory sequence that controls expression of the polynucleotide in a host cell.

11. A genetically-engineered host cell containing the polynucleotide of claim 1, 3, or 5, or progeny thereof.

12. A genetically-engineered host cell containing the polynucleotide of claim 1, 3, or 5 in which the nucleotide sequence of the polynucleotide is operatively associated with a regulatory sequence that controls expression of the polynucleotide in a host cell, or progeny thereof.

13. The host cell of claim 12 which is a prokaryote.

14. The host cell of claim 12 which is an eukaryote.

15. A method for producing a polypeptide comprising recovering the polypeptide from the genetically-engineered host cell of claim 12.

16. An isolated polypeptide comprising the amino acid sequence as shown in SEQ ID NO:2.

17. An isolated polypeptide comprising an amino acid sequence selected from the group consisting of residues 173 to 520, residues 206 to 214, residues 283 to 297, residues 438 to 451, and residues 464 to 476 of SEQ ID NO:2.

18. An isolated naturally-occurring polypeptide encoded by a polynucleotide that hybridizes under stringent conditions to a second polynucleotide comprising a nucleotide sequence which is complementary to a nucleotide sequence that encodes the amino acid sequence as shown in SEQ ID NO:2.

19. The polypeptide of claim 18 in which the second polynucleotide comprises a nucleotide sequence which is complementary to the nucleotide sequence as shown in SEQ ID NO:1.

20. The polypeptide of claim 17 which is produced by a recombinant DNA method.

21. The polypeptide of claim 17 which is fused with a heterologous polypeptide.

22. An antibody which specifically binds to a polypeptide comprising the amino acid sequence as shown in SEQ ID NO:2, or a fragment of the antibody that binds said polypeptide.

23. The fragment of the antibody of claim 22 which is a Fab, a (Fab′)₂, a Fv, a CDR or a single chain Fv.

24. The antibody of claim 22 which is a monoclonal antibody.

25. An isolated polynucleotide, comprising a nucleotide sequence that encodes a polypeptide comprising the amino acid sequence as shown in SEQ ID NO:4.

26. The polynucleotide of claim 25 in which the nucleotide sequence is shown in SEQ ID NO:3.

27. An isolated polynucleotide, comprising a nucleotide sequence that encodes a polypeptide comprising an amino acid sequence selected from the group consisting of residues 158 to 474, residues 138 to 474, residues 138 to 158, and residues 203 to 217 of SEQ ID NO:4.

28. The polynucleotide of claim 27 in which the nucleotide sequence is selected from the group consisting of residues 672 to 1622, residues 612 to 1622, residues 612 to 674, and residues 807 to 851 of SEQ ID NO:3.

29. An isolated polynucleotide, comprising a nucleotide sequence of at least 15 nucleotides that hybridizes under stringent conditions to a second polynucleotide having a nucleotide sequence as shown in SEQ ID NO:3 or to the complementary sequence of the second polynucleotide.

30. An isolated polynucleotide, comprising a nucleotide sequence that hybridizes under stringent conditions to a second polynucleotide having a nucleotide sequence as shown in SEQ ID NO:3 or the complementary sequence of the second polynucleotide, and which isolated polynucleotide encodes a naturally-occurring polypeptide.

31. The polynucleotide of claim 25, 27, or 29 which is DNA.

32. The polynucleotide of claim 25, 27, or 29 which is RNA.

33. A recombinant vector containing the polynucleotide of claim 25, 27, or 29.

34. A recombinant expression vector containing the polynucleotide of claim 25, 27, or 29 in which the nucleotide sequence of the polynucleotide is operatively associated with a regulatory sequence that controls expression of the polynucleotide in a host cell.

35. A genetically-engineered host cell containing the polynucleotide of claim 25, 27, or 29, or progeny thereof.

36. A genetically-engineered host cell containing the polynucleotide of claim 25, 27, or 29 in which the nucleotide sequence of the polynucleotide is operatively associated with a regulatory sequence that controls expression of the polynucleotide in a host cell, or progeny thereof.

37. The host cell of claim 36 which is a prokaryote.

38. The host cell of claim 36 which is an eukaryote.

39. A method for producing a polypeptide comprising recovering the polypeptide from the genetically-engineered host cell of claim 36.

40. An isolated polypeptide comprising the amino acid sequence as shown in SEQ ID NO:4.

41. An isolated polypeptide comprising an amino acid sequence selected from the group consisting of 158 to 474, residues 138 to 474, residues 138 to 158, and residues 203 to 217 of SEQ ID NO:4.

42. An isolated naturally-occurring polypeptide encoded by a polynucleotide that hybridizes under stringent conditions to a second polynucleotide comprising a nucleotide sequence which is complementary to a nucleotide sequence that encodes the amino acid sequence as shown in SEQ ID NO:4.

43. The polypeptide of claim 42 in which the second polynucleotide comprises a nucleotide sequence which is complementary to the nucleotide sequence as shown in SEQ ID NO:3.

44. The polypeptide of claim 41 which is produced by a recombinant DNA method.

45. The polypeptide of claim 41 which is fused with a heterologous polypeptide.

46. An antibody which specifically binds to a polypeptide comprising the amino acid sequence as shown in SEQ ID NO:4, or a fragment of the antibody that binds said polypeptide.

47. The fragment of the antibody of claim 46 which is a Fab, a (Fab′)₂, a Fv, a CDR or a single chain Fv.

48. The antibody of claim 46 which is a monoclonal antibody.

49. A method of screening for an inhibitor of mRNA capping, the method comprising
exposing a fungal capping reaction to a test substance, and
assaying for a change in the efficiency of the capping reaction in the presence of the test substance.

50. The method of claim 49 wherein the capping reaction comprises a C. albicans CET1 capping enyzme.

51. The method of claim 49 wherein the capping reaction comprises a C. albicans ABD1 capping enzyme.

52. The method of any one of claims 49, 50 and 51, wherein the capping reaction is performed in vitro.

53. The method of any one of claims 49, 50 and 51, wherein the capping reaction is performed in vivo.

54. The method of claim 49, wherein the fungal capping reaction comprises at least one recombinantly produced capping enzyme.

55. The method of claim 54, wherein the recombinantly produced capping enzyme is C. albicans CET1 capping enzyme.

56. The method of claim 54, wherein the recombinantly produced capping enzyme is C. albicans ABD1 capping enzyme.

57. The method of claim 49 further comprising the step of performing a mammalian capping reaction.

58. A method of screening for compounds that inhibit a fungal capping enzyme, comprising
exposing a fungal capping reaction containing a C. albicans CET1 capping enzyme to a test substance, and
assaying for the inhibition of the fungal capping reaction in the presence of the test substance relative to the absence of the test substance.

59. A method of screening for compounds that inhibit a fungal capping enzyme, comprising
exposing a fungal capping reaction containing a C. albicans ABD1 capping enzyme to a test substance, and
assaying for the inhibition of the fungal capping reaction in the presence of the test substance relative to the absence of the test substance.

60. A method of screening for compounds that bind to a capping enzyme, comprising exposing to a test substance a protein or peptide containing an amino acid sequence corresponding to at least 6 consecutive amino acids of a C. albicans CET1 capping enzyme, and assaying for the binding of the test substance to the protein or peptide.

61. A method of screening for compounds that bind to a capping enzyme, comprising exposing to a test substance a protein or peptide containing an amino acid sequence corresponding to at least 6 consecutive amino acids of a C. albicans ABD1 capping enzyme, and assaying for the binding of the test substance to the protein or peptide.

62. An assay for identifying a substance that inhibits the specific interaction of a fungal cell molecule with a fungal capping enzyme, comprising:
(a) contacting a protein or peptide containing an amino acid sequence corresponding to the binding site of the fungal cell molecule with a protein or peptide having an amino acid sequence corresponding to the binding site of the fungal capping enzyme, under conditions and for a time sufficient to permit binding and the formation of a complex, in the presence of a test substance, and
(b) detecting the formation of a complex, in which the ability of the test substance to inhibit the interaction between the fungal cell molecule and the fungal capping enzyme is indicated by a decrease in complex formation as compared to the amount of complex formed in the absence of the test substance.

63. The method of claim 62 wherein the fungal cell molecule is an RNA.

64. The method of claim 62 wherein the fungal cell molecule is a guanyltransferase and the fungal capping enzyme is a triphosphatase.

65. The method of claim 62 wherein the fungal capping enzyme is C. albicans CET1.

66. The method of claim 62 wherein the fungal capping enzyme is C. albicans ABD1.

67. A commercial kit comprising the polypeptide of claim 20.

68. The commercial kit of claim 67 further comprising a buffer or a buffer concentrate suitable for performing an mRNA capping reaction.

69. A commercial kit comprising the polypeptide of claim 44.

70. The commercial kit of claim 69 further comprising a buffer or a buffer concentrate suitable for performing an mRNA capping reaction.

71. A fungal capping reaction comprising a recombinantly produced fungal guanyltransferase, a recombinantly produced fungal triphosphatase, and a recombinantly produced fungal methyltransferase.

72. The fungal capping reaction of claim 71, wherein the recombinantly produced fungal triphosphatase is a C. albicans triphosphatase.

73. The fungal capping reaction of claim 72, wherein the C. albicans triphosphatase has the amino acid sequence presented by SEQ ID NO:2.

74. The fungal capping reaction of claim 71, wherein the recombinantly produced fungal methyltransferase is a C. albicans methyltransferase.

75. The fungal capping reaction of claim 74, wherein the C. albicans methyltransferase has the amino acid sequence presented by SEQ ID NO:4.

76. The fungal capping reaction of claim 71, wherein the fungal capping reaction is performed in vitro.
